SEQUENCE LISTING (73 sequences)

SEQ ID NO: 1 - protein, 245 aa, Homo sapiens
MEGPRGWLVLCVLAISLASMVTEDLCRAPDGKKGEAGRPGRRGRPGLKGEQGEPGAPGIRTGIQGLKGDQGEPGPSGNPGKVGYPGPSGPLGARGIPGIKGTKGSPGNIKDQPRPAFSAIRRNPPMGGNVVIFDTVITNQEEPYQNHSGRFVCTVPGYYYFTFQVLSQWEICLSIVSSSRGQVRRSLGFCDTTNKGLFQVVSGGMVLQLQQGDQVWVEKDPKKGHIYQGSEADSVFSGFLIFPSA

SEQ ID NO: 2 - protein, 253 aa, Homo sapiens
MMMKIPWGSIPVLMLLLLLGLIDISQAQLSCTGPPAIPGIPGIPGTPGPDGQPGTPGIKGEKGLPGLAGDHGEFGEKGDPGIPGNPGKVGPKGPMGPKGGPGAPGAPGPKGESGDYKATQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPDMEA

SEQ ID NO: 3 - protein, 245 aa, Homo sapiens
MDVGPSSLPHLGLKLLLLLLLLPLRGQANTGCYGIPGMPGLPGAPGKDGYDGLPGPKGEPGIPAIPGIRGPKGQKGEPGLPGHPGKNGPMGPPGMPGVPGPMGIPGEPGEEGRYKQKFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD

SEQ ID NO: 4 - protein, 107 aa, synthetic construct
DVQITQSPSYLAASPGETITINCRASKSINKYLAWYQEKPGKTNKLLIYSGSTLQSGIPSRFSGSGSGTDFTLTISSLEPEDFAMYYCQQHNEYPLTFGAGTKLELK

SEQ ID NO: 5 - protein, 11 aa, synthetic construct
RASKSINKYLA

SEQ ID NO: 6 - protein, 7 aa, synthetic construct
SGSTLQS

SEQ ID NO: 7 - protein, 9 aa, synthetic construct
QQHNEYPLT

SEQ ID NO: 8 - protein, 121 aa, synthetic construct
QVQLQQPGAELVKPGASVKLSCKSSGYHFTSYWMHWVKQRPGQGLEWIGVIHPNSGSINYNEKFESKATLTVDKSSSTAYMQLSSLTSEDSAVYYCAGERDSTEVLPMDYWGQGTSVTVSS

SEQ ID NO: 9 - protein, 10 aa, synthetic construct
GYHFTSYWMH

SEQ ID NO: 10 - protein, 17 aa, synthetic construct
VIHPNSGSINYNEKFES

SEQ ID NO: 11 - protein, 12 aa, synthetic construct
ERDSTEVLPMDY

SEQ ID NO: 12 - DNA, 321 bp, synthetic construct
  source 1..321: mol_type = other DNA
gatgtccagataacccagtctccatcttatcttgctgcatctcctggagaaaccattactattaattgcagggcaagtaagagcattaacaaatatttagcctggtatcaagagaaacctgggaaaactaataagcttcttatctactctggatccactttgcaatctggaattccatcaaggttcagtggcagtggatctggtacagatttcactctcaccatcagtagcctggagcctgaagattttgcaatgtattactgtcaacaacataatgaatacccgctcacgttcggtgctgggaccaagctggagctgaaa

SEQ ID NO: 13 - DNA, 363 bp, synthetic construct
  source 1..363: mol_type = other DNA
caggtccaactgcagcagcctggggctgagctggtaaagcctggggcttcagtgaagttgtcctgcaagtcttctggctaccatttcaccagctactggatgcactgggtgaagcagaggcctggacaaggccttgagtggattggagtgattcatcctaatagtggtagtattaactacaatgagaagttcgagagcaaggccacactgactgtagacaaatcctccagcacagcctacatgcaactcagcagcctgacatctgaggactcggcggtctattattgtgcaggagagagagattctacggaggttctccctatggactactggggtcaaggaacctcagtcaccgtctcctca

SEQ ID NO: 14-30 - skipped (17 entries)

SEQ ID NO: 31 - protein, 121 aa, synthetic construct [sequence omitted]

SEQ ID NO: 32 - protein, 121 aa, synthetic construct [sequence omitted]

SEQ ID NO: 33 - protein, 121 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSS

SEQ ID NO: 34 - protein, 121 aa, synthetic construct [sequence omitted]

SEQ ID NO: 35 - protein, 107 aa, synthetic construct [sequence omitted]

SEQ ID NO: 36 - protein, 107 aa, synthetic construct [sequence omitted]

SEQ ID NO: 37 - protein, 107 aa, synthetic construct
DVQITQSPSSLSASLGERATINCRASKSINKYLAWYQQKPGKAPKLLIYSGSTLQSGIPARFSGSGSGTDFTLTISSLEPEDFAMYYCQQHNEYPLTFGQGTKLEIK

SEQ ID NO: 38 - protein, 107 aa, synthetic construct [sequence omitted]

SEQ ID NO: 39 - protein, 229 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 40 - protein, 214 aa, synthetic construct
DVQITQSPSSLSASLGERATINCRASKSINKYLAWYQQKPGKAPKLLIYSGSTLQSGIPARFSGSGSGTDFTLTISSLEPEDFAMYYCQQHNEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 18 bp, synthetic construct
  source 1..18: mol_type = other DNA
tgcatcctgccatctcct

SEQ ID NO: 42 - DNA, 23 bp, synthetic construct
  source 1..23: mol_type = other DNA
gaaagtgcttaaagaaaccactg

SEQ ID NO: 43 - DNA, 20 bp, synthetic construct
  source 1..20: mol_type = other DNA
gcatttctggggattgctta

SEQ ID NO: 44 - DNA, 20 bp, synthetic construct
  source 1..20: mol_type = other DNA
cccggcaaaacaggtagtta

SEQ ID NO: 45 - DNA, 19 bp, synthetic construct
  source 1..19: mol_type = other DNA
aagttcatctgcaccaccg

SEQ ID NO: 46 - DNA, 20 bp, synthetic construct
  source 1..20: mol_type = other DNA
tccttgaagaagatggtgcg

SEQ ID NO: 47 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - DNA, 20 bp, synthetic construct
  source 1..20: mol_type = other DNA
tgccagtttctttggctctt

SEQ ID NO: 49 - DNA, 20 bp, synthetic construct
  source 1..20: mol_type = other DNA
tctcagccattcggcagaac

SEQ ID NO: 50 - DNA, 21 bp, synthetic construct
  source 1..21: mol_type = other DNA
tggttggtgaggaccttgtca

SEQ ID NO: 51 - DNA, 20 bp, synthetic construct
  source 1..20: mol_type = other DNA
tccggctcctgctcctctta

SEQ ID NO: 52 - DNA, 24 bp, synthetic construct
  source 1..24: mol_type = other DNA
gtatgcagctgacttcagggatgt

SEQ ID NO: 53 - DNA, 18 bp, synthetic construct
  source 1..18: mol_type = other DNA
gcccacagccttctacac

SEQ ID NO: 54 - DNA, 18 bp, synthetic construct
  source 1..18: mol_type = other DNA
ccagggtcaccatttctc

SEQ ID NO: 55 - DNA, 18 bp, synthetic construct
  source 1..18: mol_type = other DNA
tgcctcgggaatggaaag

SEQ ID NO: 56 - DNA, 24 bp, synthetic construct
  source 1..24: mol_type = other DNA
atggtaggtcttcccatcgtcata

SEQ ID NO: 57 - DNA, 21 bp, synthetic construct
  source 1..21: mol_type = other DNA
tcgccttccccaggtcccttc

SEQ ID NO: 58 - DNA, 20 bp, synthetic construct
  source 1..20: mol_type = other DNA
gccgtggtccacaccccgat

SEQ ID NO: 59 - DNA, 20 bp, synthetic construct
  source 1..20: mol_type = other DNA
cgagcggagcgacgaggagt

SEQ ID NO: 60 - DNA, 21 bp, synthetic construct
  source 1..21: mol_type = other DNA
tgggcgggatggcattttcgg

SEQ ID NO: 61 - DNA, 24 bp, synthetic construct
  source 1..24: mol_type = other DNA
aactggaaagaatgtcttgattgt

SEQ ID NO: 62 - DNA, 25 bp, synthetic construct
  source 1..25: mol_type = other DNA
gaatttcaaatccaacaaagtctgg

SEQ ID NO: 63 - DNA, 21 bp, synthetic construct
  source 1..21: mol_type = other DNA
gggaatccactgctgtccggc

SEQ ID NO: 64 - DNA, 22 bp, synthetic construct
  source 1..22: mol_type = other DNA
ctcagcctcaggggcttcctgt

SEQ ID NO: 65 - DNA, 23 bp, synthetic construct
  source 1..23: mol_type = other DNA
agagccaggaatcccagccgtcc

SEQ ID NO: 66 - DNA, 19 bp, synthetic construct
  source 1..19: mol_type = other DNA
gcatgccaggctcgccctt

SEQ ID NO: 67 - DNA, 24 bp, synthetic construct
  source 1..24: mol_type = other DNA
aaccatattacaagatgctgacca

SEQ ID NO: 68 - DNA, 18 bp, synthetic construct
  source 1..18: mol_type = other DNA
ccttgggctgtgcaggta

SEQ ID NO: 69 - DNA, 22 bp, synthetic construct
  source 1..22: mol_type = other DNA
gaccagaggcaggagaggaggc

SEQ ID NO: 70 - DNA, 22 bp, synthetic construct
  source 1..22: mol_type = other DNA
gctcagtgtcaccttcaggagc

SEQ ID NO: 71 - DNA, 21 bp, synthetic construct
  source 1..21: mol_type = other DNA
tcagaccgctttttgccgcga

SEQ ID NO: 72 - DNA, 23 bp, synthetic construct
  source 1..23: mol_type = other DNA
atcgctaatcacgacgctgggac

SEQ ID NO: 73 - DNA, 20 bp, synthetic construct
  source 1..20: mol_type = other DNA
cagagggacaggaaccactc

(19) United States
(12) Patent Application Publication
(10) Pub. No.: US 2025/0230225 A1
(43) Pub. Date: Jul. 17, 2025

(54) COMPOSITIONS AND METHODS FOR TREATING MUSCULAR DYSTROPHY

(71) Applicants: Annexon, Inc., Brisbane, CA (US); Universita’ Degli Studi Di Trento, Trento (IT); FONDAZIONE TELETHON ETS, Rome (IT)

(72) Inventors: Stefano Biressi, Povo (IT); Francesca Florio, Povo (IT); Yaisa Andrews-Zwilling, Brisbane, CA (US)

(21) Appl. No.: 18/703,211

(22) PCT Filed: Oct. 21, 2022
(86) PCT No.: PCT/US22/78510

§ 371 Date: Apr. 19, 2024

Related U.S. Application Data
(60) Provisional application No. 63/270,352, filed on Oct. 21, 2021.

(51) Int. Cl. C07K 16/18; A61K 39/00; A61P 21/00

(52) U.S. Cl. CPC C07K 16/18; A61P 21/00; A61K 2039/505

(57) ABSTRACT

The present disclosure relates generally to methods of preventing, reducing risk of developing, or treating Duchenne muscular dystrophy, Becker muscular dystrophy, Limb-Girdle Muscular Dystrophies (LGMD), Collagen Type VI-Related Disorders, Congenital Muscular Dystrophies (CMD) and Congenital Myopathies, Distal Muscular Dystrophies/Myopathies. The method comprises administering to a subject an inhibitor of the classical complement pathway, such as a C1 complex inhibitor, a C1 complex inhibitor, a C1q inhibitor, a C1s inhibitor, or a C1r inhibitor.

### RELATED APPLICATIONS

This patent application claims priority to U.S. Provisional Patent Application No. 63/270,352, filed Oct. 21, 2021, which is hereby incorporated by reference in its entirety.

### BACKGROUND

Muscular dystrophies are a group of diseases that make muscles weaker and less flexible over time. Duchenne muscular dystrophy (DMD) is the most common type. It is caused by flaws in the gene that controls how the body keeps muscles healthy.

Duchenne muscular dystrophy (DMD) is the most common inherited muscle disease of childhood. Although under normal conditions muscle stem cells (satellite cells) present a very powerful regenerative response, the regenerative potential is progressively lost in patients affected by DMD and fibrotic tissue progressively replaces muscle fibers leading to an impairment of muscle function.

Despite efforts from researchers and medical professionals worldwide who have been trying to address muscular dystrophies, and despite the promise of genome engineering approaches, there remains a critical need for developing safe and effective treatments for muscular dystrophies. Thus, there is a need for new therapies to prevent, reduce the risk of developing, and treat muscular dystrophies.

Muscular dystrophies comprise a heterogeneous group of genetic disorders characterized by progressive muscle wasting and weakness. In muscular dystrophies, muscle dysfunction arise from the mutations of genes encoding for different cellular components, including proteins associated with the sarcolemma, extracellular matrix, nuclear membrane, and sarcomeric apparatus. Different forms of dystrophy differ in terms of age of onset, severity of symptomatic progression, and distribution of affected muscles.

With an incidence of ˜1 in 5,000 male newborns, Duchenne muscular dystrophy (DMD, OMIM 310200) is the most frequent and one of the most severe forms of muscular dystrophy. DMD patients typically present during childhood with progressive weakness of limb muscles, trunk muscles, and the diaphragm, ultimately leading to wasting, kyphoscoliosis, and severe respiratory problems. A rarer (˜1 in 20,000 male births) and clinically milder form of dystrophy, Becker muscular dystrophy (BMD, OMIM 300376) has the same causative allele as DMD. DMD and BMD are caused by mutations in the DMD gene encoding for dystrophin.

Dystrophin is a component of a plasma membrane associated complex, called the dystrophin glycoprotein complex (DGC), which acts as a framework to connect the intracellular cytoskeleton to the surrounding extracellular matrix. The DGC's crucial role for proper muscle functionality and integrity is demonstrated by the overlap in pathological features between DMD/BMD and a number of dystrophies caused by mutations in genes encoding other components of the DGC: Limb-Girdle Muscular Dystrophies (LGMD) (including Sarcoglycanopathies, Dystroglycanopathies and Dysferlinopathies), Collagen Type VI-Related Disorders (including Bethlem myopathy and Ullrich congenital muscular dystrophy (UCMD)), Congenital Muscular Dystrophies (CMD) and Congenital Myopathies, Distal Muscular Dystrophies/Myopathies (including Miyoshi myopathies).

Myotonic muscular dystrophy, Facioscapulohumeral Muscular Dystrophy, Emery-Dreifuss Muscular Dystrophy, Oculopharyngeal Muscular Dystrophy appear to have a molecular etiology not attributable to alterations of the DGC, and this is reflected by specific pathological aspects. Particularly, Myotonic dystrophy (DM) is the most common adult muscular dystrophy, characterized by autosomal dominant progressive myopathy, myotonia and multiorgan involvement. To date two distinct forms have been identified: Myotonic dystrophy type 1 (DM1, Steinert's disease) that is caused by a (CTG)n expansion in DMPK, and myotonic dystrophy type 2 (DM2) that is caused by a (CCTG)n expansion in ZNF9/CNBP. Mutant transcripts aggregate as nuclear foci that sequester RNA-binding proteins, resulting in spliceopathy of downstream effector genes.

### SUMMARY

The present disclosure is generally directed to methods of preventing, reducing risk of developing, slowing or blocking progression of, or treating Duchenne muscular dystrophy, Becker muscular dystrophy, Limb-Girdle Muscular Dystrophies (LGMD) (such as Sarcoglycanopathies, Dystroglycanopathies and Dysferlinopathies), Collagen Type VI-Related Disorders (such as Bethlem myopathy and Ullrich congenital muscular dystrophy (UCMD)), Congenital Muscular Dystrophies (CMD) and Congenital Myopathies, and Distal Muscular Dystrophies/Myopathies (such as Miyoshi myopathies). The methods may comprise administering to a subject an inhibitor of the classical complement pathway, such as a C1 complex inhibitor, a C1q inhibitor, a C1s inhibitor, or a C1r inhibitor. In some aspects, provided herein is a method of preventing, reducing risk of developing, slowing or blocking progression of, or treating Duchenne muscular dystrophy. The method may comprise administering to a subject an inhibitor of the classical complement pathway.

In some embodiments, the inhibitor of the classical complement pathway is a C1 complex inhibitor, such as an antibody, a peptide, a protein, a nucleic acid, a small molecule, a gene editing agent, a base editing agent, or an epigenetic editing agent. The nucleic acid may be an antisense oligonucleotide, a miRNA, a miRNA inhibitor, an mRNA, an aptamer, or an antisense nucleic acid. The antibody may be an anti-C1 complex antibody, which preferably inhibits C1r or C1s activation or prevents their ability to act on C2 or C4, and/or binds to a combinatorial epitope within the C1 complex, wherein said combinatorial epitope comprises amino acids of both C1q and C1s; both C1q and C1r; both C1r and C1s; or each of C1q, C1r, and C1s.

In some embodiments, the inhibitor of the classical complement pathway is a C1q inhibitor, such as an antibody, a peptide, a protein, a nucleic acid, a small molecule, a gene editing agent, a base editing agent, or an epigenetic editing agent. The nucleic acid may be an antisense oligonucleotide, a miRNA, a miRNA inhibitor, an mRNA, an aptamer, or an antisense nucleic acid. The antibody may be an anti-C1q antibody, which preferably inhibits the interaction between C1q and an autoantibody or between C1q and C1r, or between C1q and C1s, and/or promotes clearance of C1q from circulation or a tissue. In some embodiments, the anti-C1q antibody has a dissociation constant (KD) that ranges from 100 nM to 0.005 nM or less than 0.005 nM, binds C1q with a binding stoichiometry that ranges from 20:1 to 1.0:1 or less than 1.0:1, and/or binds C1q with a binding stoichiometry that ranges from 6:1 to 1.0:1 or less than 1.0:1, binds C1q with a binding stoichiometry that ranges from 2.5:1 to 1.0:1 or less than 1.0:1. In some embodiments, the anti-C1q antibody specifically binds to and neutralizes a biological activity of C1q, such as (1) C1q binding to an autoantibody, (2) C1q binding to C1r, (3) C1q binding to C1s, (4) C1q binding to IgM, (5) C1q binding to phosphatidylserine, (6) C1q binding to pentraxin-3, (7) C1q binding to C-reactive protein (CRP), (8) C1q binding to globular C1q receptor (gC1qR), (9) C1q binding to complement receptor 1 (CR1), (10) C1q binding to beta-amyloid, (11) C1q binding to calreticulin, (12) C1q binding to apoptotic cells, or (13) C1q binding to B cells, and/or (1) activation of the classical complement activation pathway, (2) activation of antibody and complement dependent cytotoxicity, (3) CH50 hemolysis, (4) synapse loss, (5) B-cell antibody production, (6) dendritic cell maturation, (7) T-cell proliferation, (8) cytokine production (9) microglia activation, (10) immune complex formation, (11) phagocytosis of synapses or nerve endings, (12) activation of complement receptor 3 (CR3/C3) expressing cells or (13) neuroinflammation. CH50 hemolysis may comprise human CH50 hemolysis. In some embodiments, the antibody is capable of neutralizing from at least about 50%, to about 100% of human CH50 hemolysis, and/or the antibody is capable of neutralizing at least 50% of CH50 hemolysis at a dose of less than 150 ng/ml, less than 100 ng/ml, less than 50 ng/ml, or less than 20 ng/ml. The antibody may be a monoclonal antibody, a polyclonal antibody, a recombinant antibody, a humanized antibody, a human antibody, a chimeric antibody, a monovalent antibody, a multispecific antibody, an antibody fragment, or antibody derivative thereof. In some embodiments, the antibody is an antibody fragment and the antibody fragment is a Fab fragment, a Fab′ fragment, a F(ab′)2 fragment, a Fv fragment, a diabody, or a single chain antibody molecule.

In some embodiments, the antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7 and/or a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11. In some embodiments, the antibody comprises a light chain variable domain comprising an amino acid sequence with at least about 95% homology to the amino acid sequence selected from SEQ ID NO: 4 and 35-38 and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7, preferably the light chain variable domain comprising an amino acid sequence selected from SEQ ID NO: 4 and 35-38. In some embodiments, the antibody comprises a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11, preferably the heavy chain variable domain comprising an amino acid sequence selected from SEQ ID NO: 8 and 31-34. In some embodiments, the antibody is an antibody fragment comprising a heavy chain Fab fragment of SEQ ID NO: 39 and a light chain Fab fragment of SEQ ID NO: 40.

In some embodiments, the inhibitor of the classical complement pathway is a C1r inhibitor, such as an antibody, a peptide, a protein, a nucleic acid, a small molecule, a gene editing agent, a base editing agent, or an epigenetic editing agent. The nucleic acid is an antisense oligonucleotide, a miRNA, a miRNA inhibitor, an mRNA, an aptamer, or an antisense nucleic acid. In some embodiments, the antibody is an anti-C1r antibody, which preferably inhibits the interaction between C1r and C1q or between C1r and C1s, or wherein the anti-C1r antibody inhibits the catalytic activity of C1r or inhibits the processing of pro-C1r to an active protease. In some embodiments, the antibody is an anti-C1r antibody having a dissociation constant (KD) that ranges from 100 nM to 0.005 nM or less than 0.005 nM, the anti-C1r antibody binds C1r with a binding stoichiometry that ranges from 20:1 to 1.0:1 or less than 1.0:1, the anti-C1r antibody binds C1r with a binding stoichiometry that ranges from 6:1 to 1.0:1 or less than 1.0:1, and/or the anti-C1r antibody binds C1r with a binding stoichiometry that ranges from 2.5:1 to 1.0:1 or less than 1.0:1. In some embodiments, the anti-C1r antibody promotes clearance of C1r from circulation or a tissue.

In some embodiments, the inhibitor of the classical complement pathway is a C1s inhibitor, such as an antibody, a peptide, a protein, a nucleic acid, a small molecule, a gene editing agent, a base editing agent, or an epigenetic editing agent. The nucleic acid may be an antisense oligonucleotide, a miRNA, a miRNA inhibitor, an mRNA, an aptamer, or an antisense nucleic acid. In some embodiments, the antibody is an anti-C1s antibody, which preferably inhibits the interaction between C1s and C1q or between C1s and C1r or between C1s and C2 or C4, or wherein the anti-C1s antibody inhibits the catalytic activity of C1s or inhibits the processing of pro-C1s to an active protease or binds to an activated form of C1s. In some embodiments, the anti-C1s antibody has a dissociation constant (KD) that ranges from 100 nM to 0.005 nM or less than 0.005 nM, the anti-C1s antibody binds C1s with a binding stoichiometry that ranges from 20:1 to 1.0:1 or less than 1.0:1, the anti-C1s antibody binds C1s with a binding stoichiometry that ranges from 6:1 to 1.0:1 or less than 1.0:1, and/or the anti-C1s antibody binds C1s with a binding stoichiometry that ranges from 2.5:1 to 1.0:1 or less than 1.0:1. In some embodiments, the anti-CIs antibody promotes clearance of CIs from circulation or a tissue.

In some aspects, provided herein is a method of preventing, reducing risk of developing, slowing or blocking progression of, or treating Duchenne muscular dystrophy, Becker Muscular Dystrophy, a Limb-Girdle Muscular Dystrophy (LGMD), a Collagen Type VI-Related Disorder, a Congenital Muscular Dystrophy (CMD) or Congenital Myopathy, or a Distal Muscular Dystrophy/Myopathy. The method may comprise administering to a subject a C1q inhibitor antibody, wherein the antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7, and a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11.

### DESCRIPTION OF THE FIGURES

FIGS. 1A-1L show the evaluation of complement proteins levels in 1 year old wild type, 1 year old dystrophic and 2 year old wild type muscles. ELISA assay of proteins of the classical complement pathway C1q, C3 and C3d. Tibialis anterior (TA), diaphragm (DIA) and quadriceps (Q) muscles from ˜1 year old wild type (WT 1 yo), ˜1 year old mdx^{4Cv} (MDX 1 yo) and ˜2 years old wild type (WT 2 yo) were used. N=3. Each sample is normalized to total protein levels by dividing by the measure of the sample using the PIERCE™ BCA Protein Assay kit (ThermoFisher 23225). Data are expressed as mean with SEM. Two-tailed unpaired t-test was applied. p>0.05: ns; p≤0.05: *; p≤0.01: **; p≤0.001: *** and p≤0.0001: ****.

FIGS. 2A-2I show that C1q and CIs complement proteins levels are increased in dystrophic muscles compared to the wild type. ELISA assay of C1q (FIGS. 2A-2F) and C1s (FIGS. 2G-2I) proteins in the diaphragm (DIA), tibialis anterior (TA) and quadriceps (Q) muscles from ˜1 month old (1 mo), ˜3 month old (3 mo) ˜ and 1 year old (1 yo) wild type (WT) and mdx^{4Cv} (MDX) mice (as indicated on the graphs). Each sample is normalized to total protein levels by dividing by the measure of the sample using the PIERCEM BCA Protein Assay kit (ThermoFisher 23225). N=3 (WT 1 mo, WT 3 mo), N=4 (WT 1 yo, MDX 1 mo, MDX 3 mo, MDX 1 yo). Data are expressed as mean with SEM. Two-tailed unpaired t-test was applied. p>0.05: ns; p≤0.05: *; p≤0.01: **; p≤0.001: *** and p≤0.0001: ****.

FIGS. 3A-3I show that C3, C3d and C4 complement proteins levels are increased in dystrophic muscles compared to the wild type. ELISA assay of C3 (FIGS. 3A-3C), C3d (FIGS. 3D-3F) and C4 (FIGS. 3G-3I) proteins in the diaphragm (DIA), tibialis anterior (TA) and quadriceps (Q) muscles from ˜1 month old (1 mo), ˜3 month old (3 mo) and ˜1 year old (1 yo) wild type (WT) and mdx^{4Cv} (MDX) mice (as indicated on the graphs). N=3 (WT 1 mo, WT 3 mo), N=4 (WT 1 yo, MDX 1 mo, MDX 3 mo, MDX 1 yo). Each sample is normalized to total protein levels by dividing by the measure of the sample using the PIERCE™ BCA Protein Assay kit (ThermoFisher 23225). Data are expressed as mean with SEM. Two-tailed unpaired t-test was applied. p>0.05: ns; p≤0.05: *; p≤0.01: **; p≤0.001: *** and p≤0.0001: ****.

FIGS. 4A-4E show that dystrophic mice, which have higher levels of complement proteins compared to the wild type are less able to exert to physical activity/show muscle weakness. FIG. 4A shows mice weight (grams) of ˜1 month (1 mo), ˜3 months (3 mo) and ˜1 year old (1 yo) wild type (WT) and mdx^{4Cv} (MDX) mice used in the behavioral test shown in (FIGS. 4B-4E). FIG. 4B shows hanging test performed on ˜1 month (1 mo), ˜3 months (3 mo) and ˜1 year (1 yo) old wild type (WT) and mdx^{4Cv} (MDX) mice. FIGS. 4C-4E shows open field test performed ˜1 month (1 mo), ˜3 months (3 mo) and ˜1 year (1 yo) old wild type (WT) and mdx^{4Cv} (MDX) mice. Total distance (m) (FIG. 4C), mean speed (m/sec) (FIG. 4D) and mobile time (secs) (FIG. 4E) were evaluated for each group of animals. N=3 (WT 1 mo, WT 3 mo), N=4 (WT 1 yo, MDX 1 mo, MDX 3 mo, MDX 1 yo). Note that the behavioral tests were performed before muscles dissection and ELISA analysis on the same mice as in FIGS. 2 and 3. Data are expressed as mean with SEM. Two-tailed unpaired t-test was applied. p>0.05: ns; p≤0.05: *; p≤0.01: **; p≤0.001: *** and p≤0.0001: ****.

FIG. 5 shows the experimental plan of anti-C1q treatment of dystrophic mice. C1q antibody treatment began at 10 weeks of age and was administered for 2 weeks in vivo in Pax7^{CreER};R26R^{YEP};mdx^{4Cv} male mice at the regimen of 2 times/week at the dosage of 100 mg/kg intra-peritoneally (i.p.). Blood samples were collected at the beginning of the pharmacological treatment and at sacrifice. At the beginning and at the end of the treatment functional parameters (i.e., maximal hanging time before exhaustion and behavioral activity such as total distance travelled and mean speed) were evaluated.

FIGS. 6A-6C show C1qa^{KO};mdx^{4Cv} mouse validation. FIG. 6A shows qPCR analysis of C1qa expression in FACS isolated macrophages (CD45+F4/80+) from hindlimb muscles of ˜3 months old Lyz^{Cre+/-}C1qa^{FL/FL};mdx^{4Cv} (C1qaKO, N=4) and ˜3 to 11 months old Lyz^{Cre+/-}C1qa^{WT/WT};mdx^{4Cv} (Cntr, N=7). Data are expressed as mean with SEM. Two-tailed unpaired t-test applied. FIGS. 6B-6C show qPCR analysis of C1qa expression in diaphragm and gastrocnemius of ˜3 months old Lyz^{Cre+/-}C1qa^{WT/WT};mdx^{4Cv} (Cntr (Cre+), N=6), Lyz^{Cre+/-}C1qa^{FL/FL};mdx^{4Cv} (C1qaKO, N=5) and wild type (WT, N=3). Data are expressed as mean with SEM. One-way anova test was applied. p>0.05: ns; p≤0.05: *; p≤0.01: **; p≤0.001: *** and p≤0.0001: ****.

FIGS. 7A-7F show behavioral test on 1 month old C1qa^{KO};mdx^{4Cv} mice and controls. FIG. 7A shows mice weight (grams) of ˜1 month Lyz^{Cre+/-}C1qa^{FL/FL};mdx^{4Cv}(C1qaKO) and Lyz^{Cre+/-}C1qa^{WT/WT};mdx^{4Cv} (CNTR). FIGS. 7B, 7C show hanging test (HT) performed on mice as in (FIG. 7A). The total hanging time (FIG. 7B) and the total hanging time normalized for the mice weight (FIG. 7C) were evaluated. FIGS. 7D-7F show open field (OF) test performed on mice as in (FIG. 7A). The total distance (cm) (FIG. 7D), the mean speed (cm/sec) (FIG. 7E) and the percentage of time mobile (FIG. 7F) were evaluated. N=9 (C1qaKO), N=8 (CNTR). The tests were repeated three times every other day. The average values of the three days for each test are shown. Data are expressed as mean with SEM. Two-tailed unpaired t-test was applied. p>0.05: ns; p≤0.05:*p≤0.01: **; p≤0.001: *** and p≤0.0001: ****.

FIGS. 8A-8F show behavioral test on 2 month old C1qa^{KO};mdx^{4Cv} mice and controls. FIG. 8A shows Mice weight (grams) of ˜2 month Lyz^{Cre+/-}C1qa^{FL/FL};mdx^{4Cv}(C1qaKO) and Lyz^{Cre+/-}C1qa^{WT/WT};mdx^{4Cv} (CNTR). FIGS. 8B, 8C show hanging test (HT) performed on mice as in (FIG. 8A). The total hanging time (FIG. 8B) and the total hanging time normalized for the mice weight (FIG. 8C) were evaluated. FIGS. 8D-8F show open field (OF) test performed on mice as in (FIG. 8A). The total distance (cm) (FIG. 8D), the mean speed (cm/sec) (FIG. 8E) and the percentage of time mobile (FIG. 8F) were evaluated. N=4. Data are expressed as mean with SEM. Two-tailed unpaired t-test was applied. p>0.05: ns; p≤0.05: *; p≤0.01: **; p≤0.001: *** and p≤0.0001: ****.

FIGS. 9A-9F show behavioral tests on 3 month old C1qa^{KO};mdx^{4Cv} mice and controls. FIG. 9A shows mice weight (grams) of ˜3 month Lyz^{Cre+/-}C1qa^{FL/FL};mdx^{4Cv}(C1qaKO) and Lyz^{Cre+/-}C1qa^{WT/WT};mdx^{4Cv} (CNTR). FIGS. 9B, 9C show hanging test (HT) performed on mice as in (FIG. 9A). The total hanging time (FIG. 9B) and the total hanging time normalized for the mice weight (FIG. 9C) were evaluated. FIGS. 9D-9F show open field (OF) test performed on mice as in (FIG. 9A). The total distance (cm) (FIG. 9D), the mean speed (cm/sec) (FIG. 9E) and the percentage of time mobile (FIG. 9F) were evaluated. N=9 (C1qaKO), N=7 (CNTR). The tests were repeated three times every other day. The average values of the three days for each test are shown. Data are expressed as mean with SEM. Two-tailed unpaired t-test was applied. p>0.05: ns; p≤0.05: *p≤0.01: **; p≤0.001: *** and p≤0.0001: ****.

FIGS. 10A-10F show behavioral tests on 3-month-old dystrophic mice treated with anti-C1q antibody. FIG. 10A shows mice weight (grams) of Pax7^{CreER};R26R^{YEP};mdx^{4Cv} mice treated with anti-C1q blocking antibody (Anti-C1q) or with the control antibody (Cntr) before (PRE) and after (POST) the treatment. FIGS. 10B, 10C show hanging test (HT) performed on mice as in (FIG. 10A). The total hanging time (FIG. 10B) and the total hanging time normalized for the mice weight (FIG. 10C) were evaluated. FIG. 10D-10F show open field (OF) test performed on mice as in (FIG. 10A). The total distance (cm) (FIG. 10D), the mean speed (cm/sec) and the percentage of time mobile (FIG. 10F) were evaluated. N=6. The tests were repeated three times every other day. The average values of the three days for each test are shown. Data are expressed as mean with SEM. Two-tailed unpaired t-test was applied. p>0.05: ns; p≤0.05: *; p≤0.01: **; p≤0.001: *** and p≤0.0001: ***.

FIGS. 11A-11E show expression of canonical Wnt signaling and fibrogenic related genes in the diaphragm of C1qa^{KO};mdx^{4Cv} mice and dystrophic mice treated with anti-C1q antibody. qPCR analysis of Tgfβ (FIG. 11A), Lgr5 (FIG. 11B), collagen1a1 (FIG. 11C), collagen3a1 (FIG. 11D) and fibronectin (FIG. 11E) genes in the diaphragm of ˜3 months Lyz^{Cre+/-}C1qa^{FL/FL};mdx^{4Cv} (C1qaKO (Cre+)), Lyz^{Cre+/-}C1qa^{WT/WT};mdx^{4Cv} (CNTR (Cre+)), wild type (WT), Pax7^{CreER};R26R^{YFP};mdx^{4Cv} mice treated with C1q blocking antibody (Anti-C1q) or with the control antibody (Cntr). N=6 (CNTR (Cre+), N=5 (C1qaKO, Cntr, Anti-C1q), N=3 (WT). Data are expressed as mean with SEM. Two-tailed unpaired t-test was applied. p>0.05: ns; p≤0.05: *; p≤0.01: **; p≤0.001: ***; p≤0.0001: ****.

FIGS. 12A-12E show expression of canonical Wnt signaling and fibrogenic related genes in the gastroenemius of C1qa^{KO};mdx^{4Cv} mice and dystrophic mice treated with anti-C1q antibody. qPCR analysis of Tgfβ (FIG. 12A), Lgr5 (FIG. 12B), collagen1a1 (FIG. 12C), collagen3a1 (FIG. 12D) and fibronectin (FIG. 12E) genes in the gastrocnemius of ˜3 months Lyz^{Cre+/-}C1qa^{FL/FL};mdx^{4Cv} (C1qaKO (Cre+)), Lyz^{Cre+/-}C1qa^{WT/WT};mdx^{4Cv} (CNTR (Cre+)), wild type (WT), Pax7^{CreER};R26R^{YFP};mdx^{4Cv} mice treated with C1q blocking antibody (Anti-C1q) or with the control antibody (Cntr). N=6 (CNTR (Cre+), N=5 (C1qaKO, Cntr, Anti-C1q), N=3 (WT). Data are expressed as mean with SEM. Two-tailed unpaired t-test was applied. p>0.05: ns; p≤0.05: *; p≤0.01: **; p≤0.001: ***; p≤0.0001: ****.

FIGS. 13A-13F show expression of canonical Wnt signaling and fibrogenic related genes in the fibro/adipogenic progenitor cells of C1qa^{KO};mdx^{4Cv} mice and dystrophic mice treated with anti-C1q antibody. qPCR analysis of Tgfβ (FIG. 13A), Lgr5 (FIG. 13B), Axin2 (FIG. 13C), collagen1a1 (FIG. 13D), collagen3a1 (FIG. 13E) and fibronectin (FIG. 13F) genes in the fibro/adipogenic progenitors of ˜3 months Lyz^{Cre+/-}C1qa^{FL};mdx^{4Cv} (C1qaKO (Cre+)), Lyz^{Cre+/-}C1qa^{WT/WT};mdx^{4Cv} (CNTR (Cre+)), wild type (WT), Pax7^{CreER};R26R^{YFP};mdx^{4Cv} mice treated with C1q blocking antibody (Anti-C1q) or with the control antibody (Cntr). N=6 (CNTR (Cre+), N=5 (C1qaKO, Cntr, Anti-C1q), N=3 (WT). Data are expressed as mean with SEM. Two-tailed unpaired t-test was applied. p>0.05: ns; p≤0.05: *; p≤0.01: **; p≤0.001: ***; p≤0.0001: ****.

FIGS. 14A-14F show expression of canonical Wnt signaling and fibrogenic related genes in the satellite cells of C1qa^{KO};mdx^{4Cv} mice and dystrophic mice treated with anti-C1q antibody. qPCR analysis of Tgfβ (FIG. 14A), Lgr5 (FIG. 14B), Axin2 (FIG. 14C), collagen1a1 (FIG. 14D), collagen3a1 (FIG. 14E) and fibronectin (FIG. 14F) genes in the satellite cells of ˜3 months Lyz^{Cre+/-}C1qa^{FL/FL};mdx^{4Cv} (C1qaKO (Cre+)), Lyz^{Cre+/-}C1qa^{WT/WT};mdx^{4Cv} (CNTR (Cre+)), wild type (WT), Pax7^{CreER};R26R^{YFP};mdx^{4Cv} mice treated with C1q blocking antibody (Anti-C1q) or with the control antibody (Cntr). N=6 (CNTR (Cre+), N=5 (C1qaKO, Cntr, Anti-C1q), N=3 (WT). Data are expressed as mean with SEM. Two-tailed unpaired t-test was applied. p>0.05: ns; p≤0.05: *; p≤0.01: **; p≤0.001: ***; p≤0.0001: ****.

FIGS. 15A-15B show creatine kinase test on serum from dystrophic C1qaKO mice and dystrophic mice treated with anti-C1q antibody. FIG. 15A shows CK activity (nmol/min/mL) measured in serum samples collected from ˜3 months Lyz^{Cre+/-}C1qa^{FL/FL};mdx^{4Cv} (C1qaKO (Cre+)), Lyz^{Cre+/-}C1qa^{WT/WT};mdx^{4Cv} (CNTR (Cre+)) and wild type (WT). FIG. 15B shows CK activity (nmol/min/mL) measured in serum samples collected from Pax7^{CreER};R26R^{YFP};mdx^{4Cv} mice treated with C1q blocking antibody (Anti-C1q) or with the control antibody (Cntr) before (PRE) and after (POST) the treatment. N=6 (CNTR (Cre+), N=5 (C1qaKO, Cntr, Anti-C1q), N=3 (WT). Data are expressed as mean with SEM. One-way ANOVA test was applied. p>0.05: ns; p≤0.05: *; p≤0.01: **; p≤0.001: ***; p≤0.0001: ****.

FIGS. 16A-16F show complement levels evaluation in the plasma collected from C1qa^{KO};mdx^{4Cv} mice and dystrophic mice treated with anti-C1q antibody. ELISA assay of proteins of the classical complement pathway (i.e., C1q, C3d and C1s), C1q-C3d immune complex (IC) and C1s-C1inhibitor complex (C1sC1inh). PK indicates the amount of C1q-blocking antibody in the samples. Plasma samples were collected from the following animals: mice treated with the anti-C1q blocking antibody (indicated as B in the graphs) before (PRE) and after (POST) the treatment, mice treated with the control antibody (indicated as A in the graphs) before (PRE) and after (POST) the treatment, Lyz^{Cre+/-}C1qa^{FL/FL};mdx^{4Cv} (C1qaKO Cre+) mice at 1 month of age and at sacrifice (3 months of age), Lyz^{Cre+/-}C1qa^{WT/WT};mdx^{4Cv}(Cntr Cre+) mice at 1 month of age and at sacrifice (3 months of age), Lyz^{Cre-/-} C1qa^{FL/FL};mdx^{4Cc} or Lyz^{Cre-/-}C1qa^{WT/FL};mdx^{4Cv} (Cntr Cre−) mice at 1 month of age and at sacrifice (3 months of age) and 3 months old wild type (WT). N=5 (A, B, C1qaKO Cre+), N=6 (Cntr Cre+), N=4 (Cntr Cre−), N=3 (WT). Data are expressed as mean with SEM.

FIGS. 17A-17G show complement levels evaluation in the diaphragm of C1qa^{KO};mdx^{4Cv} mice and dystrophic mice treated with anti-C1q antibody. ELISA assay of proteins of the classical complement pathway (i.e., C1q, C3d and C1s), C1q-C3d immune complex (IC), C1s-C1inhibitor complex (C1sC1inh) and albumin (Alb). PK indicates the amount of C1q-blocking antibody in the samples. Diaphragms were collected at sacrifice (3 months old) from the following animals: mice treated with the anti-C1q blocking antibody (indicated as B in the graphs), mice treated with the control antibody (indicated as A in the graphs), Lyz^{Cre+/-}C1qa^{FL/FL};mdx^{4Cv} (C1qaKO Cre+) mice, Lyz^{Cre+/-}C1qa^{WT/WT};mdx^{4Cv} (Cntr Cre+) mice, Lyz^{Cre-/-}C1qa^{FL/FL};mdx^{4Cv} or Lyz^{Cre-/-}C1qa^{WT/FL};mdx^{4Cv} (Cntr Cre−) mice and wild type (WT). N=4 (A, B, C1qaKO Cre+, Cntr Cre+), N=3 (Cntr Cre−, WT). Data are expressed as mean with SEM.

FIGS. 18A-18I show complement levels evaluation in the diaphragm of C1qa^{KO};mdx^{4Cv} mice and dystrophic mice treated with anti-C1q antibody (Total Protein Corrected). ELISA assay of proteins of the classical complement pathway (i.e., C1q, C3d and C1s), C1q-C3d immune complex (IC), C1s-C1inhibitor complex (C1sC1inh) and albumin (Alb). PK indicates the amount of C1q-blocking antibody in the samples. All data are expressed as protein (or protein complex)/Total Protein ratio. Diaphragms were collected at sacrifice (3 months old) from the following animals: mice treated with the anti-C1q blocking antibody (indicated as B in the graphs), mice treated with the control antibody (indicated as A in the graphs), Lyz^{Cre+/-}C1qa^{FL/FL};mdx^{4Cv} (C1qaKO Cre+) mice, Lyz^{Cre+/-}C1qa^{WT/WT};mdx^{4Cv} (Cntr Cre+) mice, Lyz^{Cre-/-}C1qa^{FL/FL};mdx^{4Cv} or Lyz^{Cre-/-}C1qa^{WT/FL};mdx^{4Cv} (Cntr Cre−) mice and wild type (WT). N=4 (A, B, C1qaKO Cre+, Cntr Cre+), N=3 (Cntr Cre−, WT). Each sample is normalized to total protein levels by dividing by the measure of the sample using the PIERCE™ BCA Protein Assay kit (ThermoFisher 23225). Data are expressed as mean with SEM.

FIGS. 19A-19G show complement levels evaluation in the gastrocnemius of C1qa^{KO};mdx^{4Cv} mice and dystrophic mice treated with anti-C1q antibody. ELISA assay of proteins of the classical complement pathway (i.e., C1q, C3d and C1s), C1q-C3d immune complex (IC), C1s-C1inhibitor complex (C1sC1inh) and albumin (Alb). PK indicates the amount of C1q-blocking antibody in the samples. Gastrocnemius muscles were collected at sacrifice (3 months old) from the following animals: mice treated with the anti-C1q blocking antibody (indicated as B in the graphs), mice treated with the control antibody (indicated as A in the graphs), Lyz^{Cre+/-}C1qa^{FL/FL};mdx^{4Cv} (C1qaKO Cre+) mice, Lyz^{Cre+/-}C1qa^{WT/WT};mdx^{4Cv} (Cntr Cre+) mice, Lyz^{Cre-/-}C1qa^{FL/FL};mdx^{4Cv} or Lyz^{Cre-/-}C1qa^{WT/FL};mdx^{4Cv} (Cntr Cre−) mice and wild type (WT). N=5 (A, B, C1qaKO Cre+), N=4 (Cntr Cre−), N=6 (Cntr Cre+), N=3 (WT). Data are expressed as mean with SEM.

FIGS. 20A-20M show complement levels evaluation in the gastroenemius of C1qa^{KO};mdx^{4Cv} mice and dystrophic mice treated with anti-C1q antibody, Total Protein Corrected. ELISA assay of proteins of the classical complement pathway (i.e., C1q, C3d and Cis), C1q-C3d immune complex (IC), C1s-C1inhibitor complex (C1sC1inh) and albumin (Alb). PK indicates the amount of C1q-blocking antibody in the samples. All data are expressed as protein (or protein complex)/Total Protein ratio. Gastrocnemius muscles were collected at sacrifice (3 months old) from the following animals: mice treated with the anti-C1q blocking antibody (indicated as B in the graphs), mice treated with the control antibody (indicated as A in the graphs), Lyz^{Cre+/-}C1qa^{FL/FL};mdx^{4Cv} (C1qaKO Cre+) mice, Lyz^{Cre+/-}C1qa^{WT/WT};mdx^{4Cv} (Cntr Cre+) mice, Lyz^{Cre-/-}C1qa^{FL/FL};mdx^{4Cv} or Lyz^{Cre-/-}C1qa^{WT/FL};mdx^{4Cv} (Cntr Cre−) mice and wild type (WT). N=5 (A, B, C1qaKO Cre+), N=4 (Cntr Cre−), N=6 (Cntr Cre+), N=3 (WT). Each sample is normalized to total protein levels by dividing by the measure of the sample using the PIERCE™ BCA Protein Assay kit (ThermoFisher 23225). Data are expressed as mean with SEM.

FIGS. 21A-21G show complement levels evaluation in the liver of C1qa^{KO};mdx^{4Cv} mice and dystrophic mice treated with anti-C1q antibody. ELISA assay of proteins of the classical complement pathway (i.e., C1q, C3d and C1s), C1q-C3d immune complex (IC), C1s-C1inhibitor complex (C1sC1inh) and albumin (Alb). PK indicates the amount of C1q-blocking antibody in the samples. Livers were collected at sacrifice (3 months old) from the following animals: mice treated with the anti-C1q blocking antibody (indicated as B in the graphs), mice treated with the control antibody (indicated as A in the graphs), Lyz^{Cre+/-}C1qa^{FL/FL};mdx^{4Cv} (C1qaKO Cre+) mice, Lyz^{Cre+/-}C1qa^{WT/WT}; mdx^{4Cv} (Cntr Cre+) mice, Lyz^{Cre-/-}C1qa^{FL/FL};mdx^{4Cv} or Lyz^{Cre-/-}C1qa^{WT/FL};mdx^{4Cv} (Cntr Cre−) mice and wild type (WT). N=5 (A, B, C1qaKO Cre+), N=4 (Cntr Cre−), N=6 (Cntr Cre+), N=3 (WT). Data are expressed as mean with SEM.

FIGS. 22A-22G show complement levels evaluation in the heart of dystrophic mice treated with anti-C1q antibody. ELISA assay of proteins of the classical complement pathway (i.e., C1q, C3d and C1s), C1q-C3d immune complex (IC), C1s-C1inhibitor complex (C1sC1inh) and albumin (Alb). PK indicates the amount of C1q-blocking antibody in the samples. Hearts were collected at sacrifice (3 months old) from mice treated with the anti-C1q blocking antibody (indicated as B in the graphs) and mice treated with the control antibody (indicated as A in the graphs); N=5. Data are expressed as mean with SEM.

FIG. 23 shows that C1 complex components' expression is enhanced in dystrophic muscles. mRNA expression of C1 complex subunits C1qa, C1qb, C1qc, C1r, C1s in hindlimb muscles of ˜1 year old wild type (WT) and mdxCv (Mdx) mice. N=3. Data are expressed as mean with SEM. Two tailed unpaired t-test was applied. p>0.05: ns; p≤0.05: *; p≤0.01: **; p≤0.001: ***; p≤0.0001: ****.

FIGS. 24A-24C show that C1q subunits are expressed by infiltrating macrophages in the skeletal muscle of dystrophic mice. FIGS. 24A-24B show qPCR analysis of C1qa (A) and C1qb (B) expression in satellite cells (SC), Macrophages (MAC) and Fibro/Adipogenic Progenitors (FAPs) isolated from hindlimb muscles of ˜1 year old wild type (WT) and mdx^{4Cv} (Mdx). FIG. 24C shows number of macrophages per mg of tissue in hindlimb muscles of ˜1 year old wild type (WT) and mdx^{4Cv}(Mdx) mice. N=3. Data are expressed as mean with SEM. Two tailed unpaired t-test was applied. p>0.05: ns; p≤0.05:*; p≤0.01: **; p≤0.001: ***; p≤0.0001: ****.

FIGS. 25A-25I show behavioral test on ˜1 year-old C1qa^{KO};mdx^{4Cv} mice and controls. FIG. 25A shows mice weight (grams) of ˜1 year old Lyz^{Cre+/-}C1qa^{FL/FL};mdx^{4Cv} (C1qaKO) and Lyz^{Cre+/-}C1qa^{WT/WT};mdx^{4Cv} (CNTR). FIGS. 25B, 25C show hanging test (HT) performed on mice as in (FIG. 25A). The total hanging time (FIG. 25B) and the total hanging time normalized for the mice weight (FIG. 25C) were evaluated. FIGS. 25D-25F show open field (OF) test performed on mice as in (FIG. 25A). The total distance (cm) (FIG. 25D), the mean speed (cm/sec) (FIG. 25E) and the percentage of time mobile (FIG. 25F) were evaluated. FIG. 25G, 25H show the two limbs grip test performed on mice as in (FIG. 25A). The maximal strength normalized for the mice weight (FIG. 25G) and the total grip time normalized for the mice weight (FIG. 25H) were evaluated. FIG. 25I shows the rotarod test performed on mice as in (FIG. 25A). The total walking time normalized for the mice weight was evaluated. N=5 (C1qaKO), N=7 (CNTR), N=2 (WT). Data are expressed as mean with SEM. Two-tailed unpaired t-test was applied. p>0.05: ns; p≤0.05: *; p≤0.01: **; p≤0.001: *** and p≤0.0001: ****.

FIG. 26 shows that C1 complex components' expression is enhanced in dystrophic muscles. The mRNA expression of C1 complex subunits C1qa, C1qb, C1qc, C1r, C1s was assessed in hindlimb muscles of ˜1 year old wild type (WT) and mdx^{4Cv} (MDX) mice (N=3). Data are expressed as mean with SEM. Two tailed unpaired t-test was applied. p>0.05: ns; p≤0.05: *; p≤0.01: **; p≤0.001.

FIG. 27 shows representative immunofluorescence of gastrocnemius of ˜1 year-old mdx^{4Cv} stained with anti-C1q, anti-Axin2 antibodies, and Hoechst. Scale bar (top images): 50 μm; scale bar (bottom images): 10 μm. The positive correlation between C1q and Axin2 intensity values in each region is shown. N=3. Spearman coefficient: r.

### DETAILED DESCRIPTION

### General

The present disclosure is generally directed to methods of preventing, reducing risk of developing, slowing or blocking progression of, or treating Duchenne muscular dystrophy (“DMD”), Becker muscular dystrophy (“BMD”), Limb-Girdle Muscular Dystrophies (LGMD) (including Sarcoglycanopathies, Dystroglycanopathies and Dysferlinopathies), Collagen Type VI-Related Disorders (including Bethlem myopathy and Ullrich congenital muscular dystrophy (UCMD)), Congenital Muscular Dystrophies (CMD) and Congenital Myopathies, and Distal Muscular Dystrophies/Myopathies (including Miyoshi myopathies).

The method comprises administering to a subject an inhibitor of the classical complement pathway, such as a C1 complex inhibitor, a C1q inhibitor, a C1s inhibitor, a C1r inhibitor, or a C1 complex inhibitor (e.g., anti-C1 complex antibody). The inhibitor may be an antibody, a peptide, a protein, a nucleic acid, a small molecule, a gene editing agent, a base editing agent, or an epigenetic editing agent. The nucleic acid may be an antisense oligonucleotide, a miRNA, a miRNA inhibitor, an mRNA, an aptamer, or an antisense nucleic acid. The inhibitor may refer to a compound having the ability to inhibit a biological function of a target biomolecule whether by decreasing the activity or the expression of the target biomolecule.

The complement system is involved in the necrosis process occurring in muscle fibers during the progression of DMD and the alternative complement pathway might directly participate to tissue regeneration. However, the role of the classical complement pathway in the progression of DMD is poorly understood and its participation in muscle regeneration to date is not known. Altered serum levels of complement proteins have been reported in compromised skeletal muscles (i.e., in aging and in muscle diseases), but the local production of complement components in the skeletal muscle has not been extensively investigated neither in physiological nor in pathological conditions.

Elevated WNT-signaling has been shown to play a detrimental role in the regenerative process and promotes the accumulation of fibrotic tissue in dystrophic muscles. However, the molecular and cellular pathways responsible for this process are poorly characterized. In addition to its role in innate immunity, the classical complement component C1q might activate the canonical WNT-signaling. In some embodiments, the disclosed methods of treating muscular dystrophy are based, in part, on the discovery that complement C1q is correlating with the enhanced activity of the WNT-signaling pathway in DMD (FIG. 27).

C1q-Wnt inhibition effectively ameliorates the dystrophic phenotype in vivo in a mouse model of Duchenne muscular dystrophy.

The RNA and protein levels of the C1 complex of the complement are 10-fold increased as early as one month of age and remain elevated up to one year of age in the dystrophic mdx^{4Cv} muscles compared to the healthy controls. The anti-C1q blocking antibody regimen used in the study described in the Examples herein effectively inhibited C1q expression in the target skeletal muscles (i.e., diaphragm and gastrocnemius). Dystrophic mice treated with anti-C1q blocking antibody exhibited an increased maximum hanging time before exhaustion compared to the dystrophic mice treated with the control antibody. A similar trend suggesting the amelioration of the maximum hanging time before exhaustion was observed in the C1qa genetically ablated dystrophic mice (i.e., C1qa^{KO};mdx^{4Cv}) compared to the controls tested at ˜1 and ˜2 months of age. This amelioration is not apparent one month later suggesting a possible transient effect exerted by genetic ablation of C1q. 1 year old C1qa^{KO};mdx^{4Cv} resisted more time performing the grip test compared to the controls and exhibited a trend toward an increased maximum hanging time before exhaustion compared to the controls. Overall, the gene expression of canonical WNT-target genes and fibrogenic genes was not reduced neither in the C1qa^{KO};mdx^{4Cv} mice nor in the dystrophic mice treated with the anti-C1q blocking antibody. A trend suggesting a reduction of the creatine kinase (CK) activity in the C1qa^{KO};mdx^{4Cv} serum compared to the controls was observed. However, no differences in the CK activity levels were observed in the dystrophic mice treated with the anti-C1q antibody compared to the controls.

First, the expression of complement levels, specifically components of the classical complement pathway, were evaluated in muscles of dystrophic and wild type mice and complement protein levels were found to be increased as early as one month of age and to remain elevated up to one year of age in the dystrophic mdx^{4Cv} muscles compared to the healthy controls.

The ELISA analysis performed on the tissues (i.e., diaphragm, gastrocnemius and liver) collected from the dystrophic mice treated with anti-C1q blocking antibody showed that the adopted regimen strongly reduced the expression of C1q to levels which are similar to the C1q levels in the C1qa genetically ablated (i.e., C1qa^{KO};mdx^{4Cv}) mice tissues. Thus, treating Pax7^{CreER};R26R^{YFP};mdx^{4Cv} mice with the anti-C1q blocking antibody for 2 weeks starting at 10 weeks of age at the regimen of 2 times/week at the dosage of 100 mg/kg intra-peritoneally (i.p.) is an effective way to deplete C1q skeletal muscle and liver expression.

After having assessed that the anti-C1q blocking antibody regimen effectively depleted C1q expression in the dystrophic skeletal muscles, we evaluated the effect of C1q depletion on mice functional parameters, on the gene expression of canonical WNT target genes and fibrogenic related genes and on the creatine kinase activity levels.

At both ˜1 month and -2 months of age we observed an improved physical activity in the C1qa^{KO};mdx^{4Cv} mice compared to the controls in the four limb hanging wire test. However, this trend appeared only transient and it was no longer observed when mice were tested at ˜3 months of age. Dystrophic mice treated with the anti-C1q antibody resulted physically more active compared to the mice treated with the control antibody in the four-limb hanging wire test.

### Definitions

As used herein the specification, “a” or “an” may mean one or more. As used herein in the claim(s), when used in conjunction with the word “comprising”, the words “a” or “an” may mean one or more than one. For example, reference to an “antibody” is a reference from one to many antibodies. As used herein “another” may mean at least a second or more.

As used herein, administration “conjointly” with another compound or composition includes simultaneous administration and/or administration at different times. Administration in conjunction also encompasses administration as a co-formulation or administration as separate compositions, including at different dosing frequencies or intervals, and using the same route of administration or different routes of administration.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g., bispecific antibodies) formed from at least two intact antibodies, antibody fragments so long as they exhibit biological activity, and antibody derivatives.

An “isolated” molecule or cell is a molecule or a cell that is identified and separated from at least one contaminant molecule or cell with which it is ordinarily associated in the environment in which it was produced. Preferably, the isolated molecule or cell is free of association with all components associated with the production environment. The isolated molecule or cell is in a form other than in the form or setting in which it is found in nature. Isolated molecules therefore are distinguished from molecules existing naturally in cells; isolated cells are distinguished from cells existing naturally in tissues, organs, or individuals. In some embodiments, the isolated molecule is an anti-C1s, anti-C1q, or anti-C1r antibody of the present disclosure. In other embodiments, the isolated cell is a host cell or hybridoma cell producing an anti-C1s, anti-C1q, or anti-C1r antibody of the present disclosure.

An “isolated” antibody is one that has been identified, separated and/or recovered from a component of its production environment (e.g., naturally or recombinantly). Preferably, the isolated polypeptide is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In certain preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. An isolated antibody includes the antibody in situ within recombinant T-cells since at least one component of the antibody's natural environment will not be present. Ordinarily, however, an isolated polypeptide or antibody will be prepared by a process including at least one purification step.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

As used herein, the term “CDR” or “complementarity determining region” is intended to mean the non-contiguous antigen binding sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein.

As used herein, the terms “CDR-L1”, “CDR-L2”, and “CDR-L3” refer, respectively, to the first, second, and third CDRs in a light chain variable region. As used herein, the terms “CDR-H1”, “CDR-H2”, and “CDR-H3” refer, respectively, to the first, second, and third CDRs in a heavy chain variable region. As used herein, the terms “CDR-1”, “CDR-2”, and “CDR-3” refer, respectively, to the first, second and third CDRs of either chain's variable region.

The term “monoclonal antibody” as used herein refers to an antibody obtained from a population of substantially homogeneous antibodies, i.e., the individual antibodies of the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, monoclonal antibodies are advantageous since they are typically synthesized by hybridoma culture, uncontaminated by other immunoglobulins. The modifier “monoclonal” indicates the character of the antibody as being obtained as a substantially homogeneous population of antibodies, and is not to be construed as requiring production of the antibody by any particular method. For example, the monoclonal antibodies to be used in accordance with the present disclosure may be made by a variety of techniques, including, for example, the hybridoma method (e.g., Kohler and Milstein., Nature, 256:495-97 (1975); Hongo et al., Hybridoma, 14 (3):253-260 (1995), Harlow et al., Antibodies: A Laboratory Manual, (Cold Spring Harbor Laboratory Press, 2d ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338(2): 299-310 (2004); Lee et al., J. Mol. Biol. 340(5):1073-1093 (2004); Fellouse, Proc. Nat'l Acad. Sci. USA 101(34):12467-472 (2004); and Lee et al., J. Immunol. Methods 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

“Full-length antibodies” are usually heterotetrameric glycoproteins of about 150,000 daltons, comprising two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (VdL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains. The terms “full-length antibody,” “intact antibody” and “whole antibody” are used interchangeably to refer to an antibody in its substantially intact form, as opposed to an antibody fragment or antibody derivative. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” or “antigen-binding fragment” or “functional fragments” of antibodies comprises a portion of an intact antibody, preferably the antigen binding and/or the variable region of the intact antibody or the F region of an antibody which retains or has modified FcR binding capability. Examples of antibody fragments include Fab, Fab′, F(ab′)₂ and Fv fragments; diabodies; and linear antibodies (see U.S. Pat. No. 5,641,870, Example 2; Zapata et al., Protein Eng. 8(10):1057-1062 (1995)). Additional examples of antibody fragments include antibody derivatives such as single-chain antibody molecules, monovalent antibodies and multispecific antibodies formed from antibody fragments

An “antibody derivative” is any construct that comprises the antigen-binding region of an antibody. Examples of antibody derivatives include single-chain antibody molecules, monovalent antibodies and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments with hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. (See, e.g., M. Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126: 330-41 (1995). Other FcRs, including those to be identified in the future, are encompassed by the term “FcR” herein. FcRs can also increase the serum half-life of antibodies.

Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants with improved or diminished binding to FcRs. See also, e.g., Shields et al., J. Biol. Chem. 9(2):6591-6604 (2001).

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Plückthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 1993/011161; WO/2009/121948; WO/2014/191493; Hollinger et al., Proc. Nat'l Acad. Sci. USA 90:6444-48 (1993).

As used herein, a “chimeric antibody” refers to an antibody (immunoglobulin) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies of interest herein include PRIMATIZED© antibodies wherein the antigen-binding region of the antibody is derived from an antibody produced by, e.g., immunizing macaque monkeys with an antigen of interest. As used herein, “humanized antibody” is a subset of “chimeric antibodies.”

“Humanized” forms of non-human (e.g., murine) antibodies are chimeric antibodies that contain minimal sequence derived from non-human immunoglobulin. In some embodiments, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat'l Acad. Sci. USA, 103:3557-3562 (2006) regarding human antibodies generated via a human B-cell hybridoma technology.

The term “hypervariable region,” “HVR,” or “HV,” when used herein refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, NJ, 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., supra). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| H1 | H31-H35 | H26-H35 | H26-H32 | H30-H35 |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

The phrase “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see United States Patent Publication No. 2010-280227).

An “amino-acid modification” at a specified position refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody is one with one or more alterations in one or more HVRs thereof that result in an improvement in the affinity of the antibody for antigen, compared to a parent antibody that does not possess those alteration(s). In some embodiments, an affinity-matured antibody has nanomolar or even picomolar affinities for the target antigen. Affinity-matured antibodies are produced by procedures known in the art. For example, Marks et al., Bio Technology 10:779-783 (1992) describes affinity maturation by VH- and VL-domain shuffling. Random mutagenesis of HVR and/or framework residues is described by, for example: Barbas et al. Proc. Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically recognizes” or “specifically binds” refers to measurable and reproducible interactions such as attraction or binding between a target and an antibody that is determinative of the presence of the target in the presence of a heterogeneous population of molecules including biological molecules. For example, an antibody that specifically or preferentially binds to a target or an epitope is an antibody that binds this target or epitope with greater affinity, avidity, more readily, and/or with greater duration than it binds to other targets or other epitopes of the target. It is also understood that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³ M⁻¹ or 10⁴ M⁻¹, sometimes about 10⁵ M⁻¹ or 10⁶ M⁻¹, in other instances about 10⁶ M⁻¹ or 10⁷ M⁻¹, about 10¹ M⁻¹ to 10⁹M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

“Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

Degrees of identity and similarity can be readily calculated. (See e.g., Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991)

As used herein, an “interaction” between a complement protein and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

A “blocking” antibody, an “antagonist” antibody, an “inhibitory” antibody, or a “neutralizing” antibody is an antibody that inhibits or reduces one or more biological activities of the antigen it binds, such as interactions with one or more proteins. In some embodiments, blocking antibodies, antagonist antibodies, inhibitory antibodies, or “neutralizing” antibodies substantially or completely inhibit one or more biological activities or interactions of the antigen.

The term “inhibitor” refers to a compound having the ability to inhibit a biological function of a target biomolecule, for example, an mRNA or a protein, whether by decreasing the activity or expression of the target biomolecule. An inhibitor may be an antibody, a peptide, a protein, a nucleic acid, a small molecule, a gene editing agent, a base editing agent, or an epigenetic editing agent. The nucleic acid may be an antisense oligonucleotide, a miRNA, a miRNA inhibitor, an mRNA, an aptamer, or an antisense nucleic acid. The term “antagonist” refers to a compound that binds to a receptor, and blocks or dampens the receptor's biological response. The term “inhibitor” may also refer to an “antagonist.”

Antibody “effector functions” refer to those biological activities attributable to the Fc region (a native sequence Fc region or amino acid sequence variant Fc region) of an antibody, and vary with the antibody isotype.

As used herein, the term “affinity” refers to the equilibrium constant for the reversible binding of two agents (e.g., an antibody and an antigen) and is expressed as a dissociation constant (KD). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. For example, a subject anti-Cis antibody binds specifically to an epitope within a complement C1s protein. “Specific binding” refers to binding with an affinity of at least about 10⁻⁷ M or greater, e.g., 5×10⁻⁷ M, 10⁻⁸ M, 5×10⁻⁸ M, and greater. “Non-specific binding” refers to binding with an affinity of less than about 10⁻⁷ M, e.g., binding with an affinity of 10⁻⁶ M, 10⁻⁵ M, 10⁻⁴ M, etc.

The term “kₒₙ”, as used herein, is intended to refer to the rate constant for association of an antibody to an antigen.

The term “k_{off}”, as used herein, is intended to refer to the rate constant for dissociation of an antibody from the antibody/antigen complex.

The term “K_{D}”, as used herein, is intended to refer to the equilibrium dissociation constant of an antibody-antigen interaction.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples, and cellular samples.

An “isolated” nucleic acid molecule is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acids encoding any polypeptides and antibodies herein that exist naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors useful in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO, or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or aralkyl. Not all linkages in a polynucleotide need be identical. The preceding description applies to all polynucleotides referred to herein, including RNA and DNA.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

A “gene editing agent” as used herein, is defined as an gene editing agent, representative examples of which include CRISPR-associated nucleases such as Cas9 and Cpf1 gRNAs, Argonaute family of endonucleases, clustered regularly interspaced short palindromic repeat (CRISPR) nucleases, zinc-finger nucleases (ZFNs), transcription activator-like effector nucleases (TALENs), meganucleases, other endo- and/or exo-nucleases. See Schiffer, 2012, J Virol 88(17):8920-8936, hereby incorporated by reference.

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene of the present invention, or a fragment thereof, short interfering RNA (siRNA), and small molecules which interfere with or inhibit expression of a target biomarker nucleic acid by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs, shRNAs, or other RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

In addition to RNAi, genome editing can be used to modulate the copy number or genetic sequence of a biomarker of interest, such as constitutive or induced knockout or mutation of a biomarker of interest, such as a complement pathway component like C1q, C1r, and/or C1s. For example, the CRISPR-Cas system can be used for precise editing of genomic nucleic acids (e.g., for creating non-functional or null mutations). In such embodiments, the CRISPR guide RNA and/or the Cas enzyme may be expressed. For example, a vector containing only the guide RNA can be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

“Piwi-interacting RNA (piRNA)” is the largest class of small non-coding RNA molecules. piRNAs form RNA-protein complexes through interactions with piwi proteins. These piRNA complexes have been linked to both epigenetic and post-transcriptional gene silencing of retrotransposons and other genetic elements in germ line cells, particularly those in spermatogenesis. They are distinct from microRNA (miRNA) in size (26-31 nt rather than 21-24 nt), lack of sequence conservation, and increased complexity. However, like other small RNAs, piRNAs are thought to be involved in gene silencing, specifically the silencing of transposons. The majority of piRNAs are antisense to transposon sequences, suggesting that transposons are the piRNA target. In mammals it appears that the activity of piRNAs in transposon silencing is most important during the development of the embryo, and in both C. elegans and humans, piRNAs are necessary for spermatogenesis. piRNA has a role in RNA silencing via the formation of an RNA-induced silencing complex (RISC).

“Aptamers” are oligonucleotide or peptide molecules that bind to a specific target molecule. “Nucleic acid aptamers” are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. “Peptide aptamers” are artificial proteins selected or engineered to bind specific target molecules. These proteins consist of one or more peptide loops of variable sequence displayed by a protein scaffold. They are typically isolated from combinatorial libraries and often subsequently improved by directed mutation or rounds of variable region mutagenesis and selection. The “Affimer protein”, an evolution of peptide aptamers, is a small, highly stable protein engineered to display peptide loops which provides a high affinity binding surface for a specific target protein. It is a protein of low molecular weight, 12-14 kDa, derived from the cysteine protease inhibitor family of cystatins. Aptamers are useful in biotechnological and therapeutic applications as they offer molecular recognition properties that rival that of the commonly used biomolecule, antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications.

“Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably, the siRNA is capable of promoting RNA interference through degradation or specific post-transcriptional gene silencing (PTGS) of the target messenger RNA (mRNA).

The term “preventing” is art-recognized, and when used in relation to a condition, such as muscular dystrophy is well understood in the art, and includes administration of a composition which reduces the frequency or severity, or delays the onset, of one or more symptoms of the medical condition in a subject relative to a subject who does not receive the composition. Thus, the prevention of muscular dystrophy includes, for example, retaining muscle strength in a population of patients receiving a therapy relative to a control population that did not receive the therapy, e.g., by a statistically and/or clinically significant amount. Similarly, the prevention of muscular dystrophy includes reducing the likelihood that a patient receiving a therapy will develop muscular dystrophy or related symptoms, relative to a patient who does not receive the therapy.

The term “slowing or blocking progression” as used herein refers to slowing the rate of or halting progression of a condition, such as muscular dystrophy. Disease progression describes the natural history of the disease and is assessed by measuring and monitoring functional outcome over a period of time. For example, as the disease progresses, muscle weakness and wasting (atrophy) progresses. Administration of a composition, described herein, may slow or block the progression of muscle weakness and wasting.

The term “subject” as used herein refers to a living mammal and may be interchangeably used with the term “patient”. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

As used herein, the term “treating” or “treatment” includes reducing, arresting, or reversing the symptoms, clinical signs, or underlying pathology of a condition to stabilize or improve a subject's condition or to reduce the likelihood that the subject's condition will worsen as much as if the subject did not receive the treatment.

The term “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment. A therapeutically effective amount herein may vary according to factors such as the disease state, age, sex, and weight of the patient, and the ability of the antibody to elicit a desired response in the individual.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

“Chronic” administration refers to administration of the medicament(s) in a continuous as opposed to acute mode, so as to maintain the initial therapeutic effect (activity) for an extended period of time. “Intermittent” administration refers to treatment that is not administered consecutively without interruption, but rather is cyclic/periodic in nature.

As used herein, administration “conjointly” with another compound or composition includes simultaneous administration and/or administration at different times. Conjoint administration also encompasses administration as a co-formulation or administration as separate compositions, including at different dosing frequencies or intervals, and using the same route of administration or different routes of administration.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### Classical Complement Inhibitor

The present disclosure is generally directed to methods of preventing, reducing risk of developing, slowing or blocking progression of, or treating Duchenne muscular dystrophy (“DMD”), Becker muscular dystrophy (“BMD”), Limb-Girdle Muscular Dystrophies (LGMD) (including Sarcoglycanopathies, Dystroglycanopathies and Dysferlinopathies), Collagen Type VI-Related Disorders (including Bethlem myopathy and Ullrich congenital muscular dystrophy (UCMD)), Congenital Muscular Dystrophies (CMD) and Congenital Myopathies, and Distal Muscular Dystrophies/Myopathies (including Miyoshi myopathies). The method comprises administering to a subject an inhibitor of the classical complement pathway, such as a C1 complex inhibitor, a C1q inhibitor, a C1s inhibitor, a C1r inhibitor, or a C1 complex inhibitor. The inhibitor may be an antibody, a peptide, a protein, a nucleic acid, a small molecule, a gene editing agent, a base editing agent, or an epigenetic editing agent. The nucleic acid may be an antisense oligonucleotide, a miRNA, a miRNA inhibitor, an mRNA, an aptamer, or an antisense nucleic acid. The inhibitor may refers to a compound having the ability to inhibit a biological function of a target biomolecule whether by decreasing the activity or expression of the target biomolecule.

The inhibitor may block activation of the complement cascade, may block the expression of specific complement proteins, may interfere with signaling molecules that induce complement activation, may upregulate expression of complement inhibitors, and otherwise interfere with the role of complement.

Based on the molecular structures of C1 complex, C1q, C1r, or C1s or the variable regions of the anti -C1q, -C1r, or -C1s antibodies, molecular modeling and rational molecular design may be used to generate and screen small molecules that mimic the molecular structures of the binding region of the antibodies and/or inhibit the activities of C1 complex, C1q, C1r, or C1s. These small molecules can be peptides, peptidomimetics, oligonucleotides, or organic compounds. The mimicking molecules can be used as inhibitors of complement activation. Alternatively, one can use large-scale screening procedures commonly used in the field to isolate suitable small molecules from libraries of combinatorial compounds.

A number of molecules are known that inhibit the activity of complement. In addition to known compounds, suitable inhibitors can be screened by methods described herein. For example, normal cells can produce proteins that block complement activity, e.g., CD59, C1 inhibitor, etc. In some embodiments of the disclosure, complement is inhibited by upregulating expression of genes encoding such polypeptides.

Modifications of molecules that block complement activation are also known in the art. For example, such molecules include, without limitation, modified complement receptors, such as soluble CR1. The mature protein of the most common allotype of CR1 contains 1998 amino acid residues: an extracellular domain of 1930 residues, a transmembrane region of 25 residues, and a cytoplasmic domain of 43 residues. The entire extracellular domain is composed of 30 repeating units referred to as short consensus repeats (SCRs) or complement control protein repeats (CCPRs), each consisting of 60 to 70 amino acid residues. Recent data indicate that C1q binds specifically to human CR1. Thus, CR1 recognizes all three complement opsonins, namely C3b, C4b, and C1q. A soluble version of recombinant human CR1 (sCR1) lacking the transmembrane and cytoplasmic domains has been produced and shown to retain all the known functions of the native CR1. Several types of human C1q receptors (C1qR) have been described. These include the ubiquitously distributed 60- to 67-kDa receptor, referred to as cC1qR because it binds the collagen-like domain of C1q. This C1qR variant was shown to be calreticulin; a 126-kDa receptor that modulates monocyte phagocytosis. gC1qR is not a membrane-bound molecule, but rather a secreted soluble protein with affinity for the globular regions of C1q, and may act as a fluid-phase regulator of complement activation.

Decay accelerating factor (DAF) (CD55) is composed of four SCRs plus a serine/threonine-enriched domain that is capable of extensive O-linked glycosylation. DAF is attached to cell membranes by a glycosyl phosphatidyl inositol (GPI) anchor and, through its ability to bind C4b and C3b, it acts by dissociating the C3 and C5 convertases. Soluble versions of DAF (sDAF) have been shown to inhibit complement activation.

C1 inhibitor, a member of the “serpin” family of serine protease inhibitors, is a heavily glycosylated plasma protein that prevents fluid-phase C1 activation. C1 inhibitor regulates the classical pathway of complement activation by blocking the active site of C1r and C1s and dissociating them from C1q.

Peptide inhibitors of complement activation include C5a and other inhibitory molecules include Fucan.

All sequences mentioned in the present disclosure are incorporated by reference from U.S. Pat. No. 10,316,081, U.S. patent application Ser. No. 14/890,811, U.S. Pat. Nos. 8,877,197, 9,708,394, 10,723,788, 9,562,106, 10,450,382, 10,457,745, International Patent Application No. PCT/US2018/022462 each of which is hereby incorporated by reference for the antibodies and related compositions that it discloses.

Anti-C1 complex Antibodies

The inhibitor of the classical complement pathway may be an anti-C1 complex antibody, optionally wherein the anti-C1 complex antibody inhibits C1r or C1s activation or prevents their ability to act on C2 or C4, e.g., the anti-C1 complex antibody binds to a combinatorial epitope within the C1 complex, wherein said combinatorial epitope comprises amino acids of both C1q and C1s; both C1q and C1r; both C1r and C1s; or each of C1q, C1r, and C1s. The antibody may be a monoclonal antibody. In some embodiments, the antibody inhibits cleavage of C4 and does not inhibit cleavage of C2, or inhibits cleavage of C2 and does not inhibit cleavage of C4.

In some embodiments, the antibody binds mammalian C1q, C1r, or C1s, or binds human C1q, C1r, or C1s. In some embodiments, the antibody binds mammalian C1 complex.

### Anti-Complement C1q Antibodies

The anti-C1q antibodies disclosed herein are potent inhibitors of C1q and can be dosed for continuous inhibition of C1q function over any period, and then optionally withdrawn to allow for return of normal C1q function at times when its activity may be important. Results obtained with anti-C1q antibodies disclosed herein in animal studies can be readily carried forward into the clinic with humanized or human antibodies, as well as with fragments and/or derivatives thereof.

C1q is a large multimeric protein of 460 kDa consisting of 18 polypeptide chains (6 C1q A chains, 6 C1q B chains, and 6 C1q C chains). C1r and C1s complement proteins bind to the C1q tail region to form the C1 complex (C1qr₂s₂).

The anti-C1q antibodies of this disclosure specifically recognize complement factor C1q and/or C1q in the C1 complex of the classical complement activation pathway. The bound complement factor may be derived, without limitation, from any organism having a complement system, including any mammalian organism such as human, mouse, rat, rabbit, monkey, dog, cat, cow, horse, camel, sheep, goat, or pig.

As used herein “C1 complex” refers to a protein complex that may include, without limitation, one C1q protein, two C1r proteins, and two C1s proteins (e.g., C1qr²s²).

Anti-C1q antibodies disclosed herein may inhibit C1 complex formation.

As used herein “complement factor C1q” refers to both wild type sequences and naturally occurring variant sequences.

A non-limiting example of a complement factor C1q recognized by antibodies of this disclosure is human C1q, including the three polypeptide chains A, B, and C:
C1q, chain A (Homo sapiens), Accession No. Protein Data Base: NP_057075.1; GenBank No.: NM_015991: >gi|7705753|ref|NP_057075.1|complement C1q subcomponent subunit A precursor [Homo sapiens]

(SEQ ID NO: 1)
MEGPRGWLVLCVLAISLASMVTEDLCRAPDGKKGEAGRPGRRGRPGLKGE
QGEPGAPGIRTGIQGLKGDQGEPGPSGNPGKVGYPGPSGPLGARGIPGIK
GTKGSPGNIKDQPRPAFSAIRRNPPMGGNVVIFDTVITNQEEPYQNHSGR
FVCTVPGYYYFTFQVLSQWEICLSIVSSSRGQVRRSLGFCDTTNKGLFQV
VSGGMVLQLQQGDQVWVEKDPKKGHIYQGSEADSVFSGFLIFPSA.

C1q, chain B (Homo sapiens), Accession No. Protein Data Base: NP 000482.3; GenBank No.: NM 000491.3: >gi|87298828|ref|NP_000482.3|complement C1q subcomponent subunit B precursor [Homo sapiens]

(SEQ ID NO: 2)
MMMKIPWGSIPVLMLLLLLGLIDISQAQLSCTGPPAIPGIPGIPGTPGPD
GQPGTPGIKGEKGLPGLAGDHGEFGEKGDPGIPGNPGKVGPKGPMGPKGG
PGAPGAPGPKGESGDYKATQKIAFSATRTINVPLRRDQTIRFDHVITNMN
NNYEPRSGKFTCKVPGLYYFTYHASSRGNLCVNLMRGRERAQKVVTFCDY
AYNTFQVTTGGMVLKLEQGENVFLQATDKNSLLGMEGANSIFSGFLLFPD
MEA.

C1q, chain C (Homo sapiens), Accession No. Protein Data Base: NP_001107573.1; GenBank No.: NM_001114101.1: >gi|166235903|refNP_001107573.1|complement C1q subcomponent subunit C precursor [Homo sapiens]

(SEQ ID NO: 3)
MDVGPSSLPHLGLKLLLLLLLLPLRGQANTGCYGIPGMPGLPGAPGKDGY
DGLPGPKGEPGIPAIPGIRGPKGQKGEPGLPGHPGKNGPMGPPGMPGVPG
PMGIPGEPGEEGRYKQKFQSVFTVTRQTHQPPAPNSLIRFNAVLTNPQGD
YDTSTGKFTCKVPGLYYFVYHASHTANLCVLLYRSGVKVVTFCGHTSKTN
QVNSGGVLLRLQVGEEVWLAVNDYYDMVGIQGSDSVFSGFLLFPD.

Accordingly, an anti-C1q antibody of the present disclosure may bind to polypeptide chain A, polypeptide chain B, and/or polypeptide chain C of a C1q protein. In some embodiments, an anti-C1q antibody of the present disclosure binds to polypeptide chain A, polypeptide chain B, and/or polypeptide chain C of human C1q or a homolog thereof, such as mouse, rat, rabbit, monkey, dog, cat, cow, horse, camel, sheep, goat, or pig C1q. In some embodiments, the anti-C1q antibody is a human antibody, a humanized antibody, a chimeric antibody, or a fragment thereof or a derivative thereof. In some embodiments, the antibody is humanized antibody. In some embodiments, the antibody is antibody fragment, such as, a Fab fragment.

### Light Chain and Heavy Chain Variable Domain Sequences of Antibody M1

All sequences mentioned related to Antibody M1 are incorporated by reference from U.S. Pat. No. 9,708,394, which is hereby incorporated by reference for the antibodies and related compositions that it discloses.

Using standard techniques, the nucleic acid and amino acid sequences encoding the light chain variable and the heavy chain variable domain of antibody M1 were determined. The amino acid sequence of the light chain variable domain of antibody M1 is:

(SEQ ID NO: 4)
DVQITQSPSYLAASPGETITINCRASKSINKYLAWYQEKPGKTNKLLIYS
GSTLQSGIPSRFSGSGSGTDFTLTISSLEPEDFAMYYCQQHNEYPLTFGA
GTKLELK.

The hyper variable regions (HVRs) of the light chain variable domain are depicted in bolded and underlined text. In some embodiments, the HVR-L1 of the M1 light chain variable domain has the sequence RASKSINKYLA (SEQ ID NO:5), the HVR-L2 of the M1 light chain variable domain has the sequence SGSTLQS (SEQ ID NO:6), and the HVR-L3 of the M1 light chain variable domain has the sequence QQHNEYPLT (SEQ ID NO:7).

The amino acid sequence of the heavy chain variable domain of antibody M1 is:

(SEQ ID NO: 8)
QVQLQQPGAELVKPGASVKLSCKSSGYHFTSYWMHWVKQRPGQGLEWIGV
IHPNSGSINYNEKFESKATLTVDKSSSTAYMQLSSLTSEDSAVYYCAGER
DSTEVLPMDYWGQGTSVTVSS.

The hyper variable regions (HVRs) of the heavy chain variable domain are depicted in bolded and underlined text. In some embodiments, the HVR-H1 of the M1 heavy chain variable domain has the sequence GYHFTSYWMH (SEQ ID NO:9), the HVR-H2 of the M1 heavy chain variable domain has the sequence VIIIPNSGSINYNEKFES (SEQ ID NO:10), and the HVR-H3 of the M1 heavy chain variable domain has the sequence ERDSTEVLPMDY (SEQ ID NO:11).

The nucleic acid sequence encoding the light chain variable domain was determined to be:

(SEQ ID NO: 12)
GATGTCCAGATAACCCAGTCTCCATCTTATCTTGCTGCATCTCCT
GGAGAAACCATTACTATTAATTGCAGGGCAAGTAAGAGCATTAAC
AAATATTTAGCCTGGTATCAAGAGAAACCTGGGAAAACTAATAAG
CTTCTTATCTACTCTGGATCCACTTTGCAATCTGGAATTCCATCA
AGGTTCAGTGGCAGTGGATCTGGTACAGATTTCACTCTCACCATC
AGTAGCCTGGAGCCTGAAGATTTTGCAATGTATTACTGTCAACAA
CATAATGAATACCCGCTCACGTTCGGTGCTGGGACCAAGCTGGAG
CTGAAA.

The nucleic acid sequence encoding the heavy chain variable domain was determined to be:

(SEQ ID NO: 13)
CAGGTCCAACTGCAGCAGCCTGGGGCTGAGCTGGTAAAGCCTGGG
GCTTCAGTGAAGTTGTCCTGCAAGTCTTCTGGCTACCATTTCACC
AGCTACTGGATGCACTGGGTGAAGCAGAGGCCTGGACAAGGCCTT
GAGTGGATTGGAGTGATTCATCCTAATAGTGGTAGTATTAACTAC
AATGAGAAGTTCGAGAGCAAGGCCACACTGACTGTAGACAAATCC
TCCAGCACAGCCTACATGCAACTCAGCAGCCTGACATCTGAGGAC
TCGGCGGTCTATTATTGTGCAGGAGAGAGAGATTCTACGGAGGTT
CTCCCTATGGACTACTGGGGTCAAGGAACCTCAGTCACCGTCTCC
TCA.

### Deposit of Material

The following materials have been deposited according to the Budapest Treaty in the American Type Culture Collection, ATCC Patent Depository, 10801 University Blvd., Manassas, Va. 20110-2209, USA (ATCC):

Deposit ATCC

| | | | Accession |
|---|---|---|---|
| Sample ID | Isotype | Date | No. |
| Mouse hybridoma C1qM1 | IgG1, | Jun. 6, | PTA-120399 |
| 7788-1(M) 051613 | kappa | 2013 | |
| producing | | | |
| anti-C1q antibody M1 | | | |

The hybridoma cell line producing the M1 antibody (mouse hybridoma C1qM1 7788-1(M) 051613) has been deposited with ATCC under conditions that assure that access to the culture will be available during pendency of the patent application and for a period of 30 years, or 5 years after the most recent request, or for the effective life of the patent, whichever is longer. A deposit will be replaced if the deposit becomes nonviable during that period. The deposit is available as required by foreign patent laws in countries wherein counterparts of the subject application, or its progeny are filed. However, it should be understood that the availability of the deposit does not constitute a license to practice the subject invention in derogation of patent rights granted by governmental action.

In some embodiments, the amino acid sequence of the light chain variable domain and heavy chain variable domain comprise one or more of SEQ ID NO:5 of HVR-L1, SEQ ID NO:6 of HVR-L2, SEQ ID NO:7 of HVR-L3, SEQ ID NO:9 of HVR-H1, SEQ ID NO:10 of HVR-H2, and SEQ ID NO:11 of HVR-H3.

The antibody may comprise a light chain variable domain amino acid sequence that is at least 85%, 90%, or 95% identical to SEQ ID NO:4, preferably while retaining the HVR-L1 RASKSINKYLA (SEQ ID NO:5), the HVR-L2 SGSTLQS (SEQ ID NO:6), and the HVR-L3 QQHNEYPLT (SEQ ID NO:7). The antibody may comprise a heavy chain variable domain amino acid sequence that is at least 85%, 90%, or 95% identical to SEQ ID NO:8, preferably while retaining the HVR-H1 GYHFTSYWMH (SEQ ID NO:9), the HVR-H2 VIHPNSGSINYNEKFES (SEQ ID NO:10), and the HVR-H3 ERDSTEVLPMDY (SEQ ID NO:11).

### Humanized Anti-Complement C1q Antibodies

Humanized antibodies of the present disclosure specifically bind to a complement factor C1q and/or C1q protein in the C1 complex of the classical complement pathway. The humanized anti-C1q antibody may specifically bind to human C1q, human and mouse C1q, to rat C1q, or human C1q, mouse C1q, and rat Cq.

All sequences related to humanized anti-C1q antibodies are incorporated by reference from U.S. Pat. No. 10,316,081, which is hereby incorporated by reference for the antibodies and related compositions that it discloses.

In some embodiments, the human heavy chain constant region is a human IgG4 heavy chain constant region comprising the amino acid sequence of SEQ ID NO:47, or with at least 70%, at least about 75%, at least about 80%, at least about 85%, at least about 90% homology to SEQ ID NO: 47. The human IgG4 heavy chain constant region may comprise an Fc region with one or more modifications and/or amino acid substitutions according to Kabat numbering. In such cases, the Fc region comprises a leucine to glutamate amino acid substitution at position 248, wherein such a substitution inhibits the Fc region from interacting with an Fc receptor. In some embodiments, the Fc region comprises a serine to proline amino acid substitution at position 241, wherein such a substitution prevents arm switching in the antibody.

The amino acid sequence of human IgG4 (S241P L248E) heavy chain constant domain is:

(SEQ ID NO: 47)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPPCPAPEFEGGPSVFLFPPKPKDTLMISR
TPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVMHEALHNH
YTQKSLSLSLGK.

The antibody may comprise a heavy chain variable domain and a light chain variable domain, wherein the heavy chain variable domain comprises an amino acid sequence selected from any one of SEQ ID NOs: 31-34, or an amino acid sequence with at least about 90% homology to the amino acid sequence selected from any one of SEQ ID NOs: 31-34. In certain such embodiments, the light chain variable domain comprises an amino acid sequence selected from any one of SEQ ID NOs: 35-38, or an amino acid sequence with at least about 90% homology to the amino acid sequence selected from any one of SEQ ID NOs: 35-38. The amino acid sequence of heavy chain variable domain variant 1 (VH1) is:

(SEQ ID NO: 31)
QVQLVQSGAELKKPGASVKVSCKSSGYHFTSYWMHWVKQAPGQGL
EWIGVIHPNSGSINYNEKFESKATITVDKSTSTAYMQLSSLTSED
SAVYYCAGERDSTEVLPMDYWGQGTSVTVSS

The hyper variable regions (HVRs) of VH1 are depicted in bolded and underlined text.

The amino acid sequence of heavy chain variable domain variant 2 (VH2) is:

(SEQ ID NO: 32)
QVQLVQSGAELKKPGASVKVSCKSSGYHFTSYWMHWVKQAPGQGL
EWIGVIHPNSGSINYNEKFESRATITVDKSTSTAYMELSSLRSED
TAVYYCAGERDSTEVLPMDYWGQGTTVTVSS.

The hyper variable regions (HVRs) of VH2 are depicted in bolded and underlined text.

The amino acid sequence of heavy chain variable domain variant 3 VH3 is:

(SEQ ID NO: 33)
QVQLVQSGAELKKPGASVKVSCKSSGYHFTSYWMHWVKQAPGQGL
EWIGVIHPNSGSINYNEKFESRVTITVDKSTSTAYMELSSLRSED
TAVYYCAGERDSTEVLPMDYWGQGTTVTVSS.

The hyper variable regions (HVRs) of VH3 are depicted in bolded and underlined text.

The amino acid sequence of heavy chain variable domain variant 4 (VH4) is:

(SEQ ID NO: 34)
QVQLVQSGAELKKPGASVKVSCKSSGYHFTSYWMHWVRQAPGQGL
EWIGVIHPNSGSINYNEKFESRVTITVDKSTSTAYMELSSLRSED
TAVYYCAGERDSTEVLPMDYWGQGTTVTVSS

The hyper variable regions (HVRs) of VH4 are depicted in bolded and underlined text.

The amino acid sequence of kappa light chain variable domain variant 1 (Vκ1) is:

(SEQ ID NO: 35)
DVQITQSPSYLAASLGERATINCRASKSINKYLAWYQQKPGKTNK
LLIYSGSTLQSGIPARFSGSGSGTDFTLTISSLEPEDFAMYYCQQ
HNEYPLTFGQGTKLEIK.

The hyper variable regions (HVRs) of Vκ1 are depicted in bolded and underlined text.

The amino acid sequence of kappa light chain variable domain variant 2 (Vκ2) is:

(SEQ ID NO: 36)
DVQITQSPSSLSASLGERATINCRASKSINKYLAWYQQKPGKANK
LLIYSGSTLQSGIPARFSGSGSGTDFTLTISSLEPEDFAMYYCQQ
HNEYPLTFGQGTKLEIK.

The hyper variable regions (HVRs) of Vκ2 are depicted in bolded and underlined text.

The amino acid sequence of kappa light chain variable domain variant 3 (Vκ3) is:

(SEQ ID NO: 37)
DVQITQSPSSLSASLGERATINCRASKSINKYLAWYQQKPGKAPK
LLIYSGSTLQSGIPARFSGSGSGTDFTLTISSLEPEDFAMYYCQQ
HNEYPLTFGQGTKLEIK.

The hyper variable regions (HVRs) of Vκ3 are depicted in bolded and underlined text.

The amino acid sequence of kappa light chain variable domain variant 4 (Vκ4) is:

(SEQ ID NO: 38)
DIQLTQSPSSLSASLGERATINCRASKSINKYLAWYQQKPGKAPK
LLIYSGSTLQSGIPARFSGSGSGTDFTLTISSLEPEDFAMYYCQQ
HNEYPLTFGQGTKLEIK.

The hyper variable regions (HVRs) of Vκ4 are depicted in bolded and underlined text.

The antibody may comprise a light chain variable domain amino acid sequence that is at least 85%, 90%, or 95% identical to SEQ ID NO:35-38 while retaining the HVR-L1 RASKSINKYLA (SEQ ID NO:5), the HVR-L2 SGSTLQS (SEQ ID NO:6), and the HVR-L3 QQHNEYPLT (SEQ ID NO:7). The antibody may comprise a heavy chain variable domain amino acid sequence that is at least 85%, 90%, or 95% identical to SEQ ID NO:31-34 while retaining the HVR-H1 GYHFTSYWMH (SEQ ID NO:9), the HVR-H2 VIHPNSGSINYNEKFES (SEQ ID NO:10), and the HVR-H3 ERDSTEVLPMDY (SEQ ID NO:11).

In some embodiments, the antibody comprises a light chain variable domain amino acid sequence of SEQ ID NO: 35 and a heavy chain variable domain amino acid sequence of SEQ ID NO: 31. In some embodiments, the antibody comprises a light chain variable domain amino acid sequence of SEQ ID NO: 36 and a heavy chain variable domain amino acid sequence of SEQ ID NO: 32. In some embodiments, the antibody comprises a light chain variable domain amino acid sequence of SEQ ID NO: 37 and a heavy chain variable domain amino acid sequence of SEQ ID NO: 33. In some embodiments, the antibody comprises a light chain variable domain amino acid sequence of SEQ ID NO: 38 and a heavy chain variable domain amino acid sequence of SEQ ID NO: 34.

In some embodiments, humanized anti-C1q antibodies of the present disclosure include a heavy chain variable region that contains a Fab region and a heavy chain constant regions that contains an Fc region, where the Fab region specifically binds to a C1q protein of the present disclosure, but the Fc region is incapable of binding the C1q protein. In some embodiments, the Fc region is from a human IgG1, IgG2, IgG3, or IgG4 isotype. In some embodiments, the Fc region is incapable of inducing complement activity and/or incapable of inducing antibody-dependent cellular cytotoxicity (ADCC). In some embodiments, the Fc region comprises one or more modifications, including, without limitation, amino acid substitutions. In certain embodiments, the Fc region of humanized anti-C1q antibodies of the present disclosure comprise an amino acid substitution at position 248 according to Kabat numbering convention or a position corresponding to position 248 according to Kabat numbering convention, and/or at position 241 according to Kabat numbering convention or a position corresponding to position 241 according to Kabat numbering convention. In some embodiments, the amino acid substitution at position 248 or a positions corresponding to position 248 inhibits the Fc region from interacting with an Fc receptor. In some embodiments, the amino acid substitution at position 248 or a positions corresponding to position 248 is a leucine to glutamate amino acid substitution. In some embodiments, the amino acid substitution at position 241 or a positions corresponding to position 241 prevents arm switching in the antibody. In some embodiments, the amino acid substitution at position 241 or a positions corresponding to position 241 is a serine to proline amino acid substitution. In certain embodiments, the Fc region of humanized anti-C1q antibodies of the present disclosure comprises the amino acid sequence of SEQ ID NO: 47, or an amino acid sequence with at least about 70%, at least about 75%, at least about 80% at least about 85% at least about 90%, or at least about 95% homology to the amino acid sequence of SEQ ID NO: 47.

### Anti-C1q Fab Fragment

Before the advent of recombinant DNA technology, proteolytic enzymes (proteases) that cleave polypeptide sequences have been used to dissect the structure of antibody molecules and to determine which parts of the molecule are responsible for its various functions. Limited digestion with the protease papain cleaves antibody molecules into three fragments. Two fragments, known as Fab fragments, are identical and contain the antigen-binding activity. The Fab fragments correspond to the two identical arms of the antibody molecule, each of which consists of a complete light chain paired with the V_{H} and C_{H}1 domains of a heavy chain. The other fragment contains no antigen binding activity but was originally observed to crystallize readily, and for this reason was named the Fc fragment (Fragment crystallizable). When Fab molecules were compared to IgG molecules, it was found that Fab are superior to IgG for certain in vivo applications due to their higher mobility and tissue penetration capability, their reduced circulatory half-life, their ability to bind antigen monovalently without mediating antibody effector functions, and their lower immunogenicity.

The Fab molecule is an artificial ˜50-kDa fragment of the Ig molecule with a heavy chain shortened by constant domains C_{H}2 and C_{H}3. Two heterophilic (V_{L}-V_{H} and C_{L}-C_{H}1) domain interactions underlie the two-chain structure of the Fab molecule, which is further stabilized by a disulfide bridge between C_{L} and C_{H}1. Fab and IgG have identical antigen binding sites formed by six complementarity-determining regions (CDRs), three each from V_{L} and V_{H} (LCDR1, LCDR2, LCDR3 and HCDR1, HCDR2, HCDR3). The CDRs define the hypervariable antigen binding site of antibodies. The highest sequence variation is found in LCDR3 and HCDR3, which in natural immune systems are generated by the rearrangement of V_{L} and J_{L} genes or V_{H}, D_{H} and J_{H} genes, respectively. LCDR3 and HCDR3 typically form the core of the antigen binding site. The conserved regions that connect and display the six CDRs are referred to as framework regions. In the three-dimensional structure of the variable domain, the framework regions form a sandwich of two opposing antiparallel β-sheets that are linked by hypervariable CDR loops on the outside and by a conserved disulfide bridge on the inside. This unique combination of stability and versatility of the antigen binding site of Fab and IgG underlie its success in clinical practice for the diagnosis, monitoring, prevention, and treatment of disease.

All anti-C1q antibody Fab fragment sequences are incorporated by reference from U.S. Pat. No. 10,723,788, which is hereby incorporated by reference for the antibodies and related compositions that it discloses.

In certain embodiments, the present disclosure provides an anti-C1q antibody Fab fragment that binds to a C1q protein comprising a heavy (V_{H}/C_{H}1) and light chain (V_{L}/C_{L}), wherein the anti-C1q antibody Fab fragment has six complementarity determining regions (CDRs), three each from V_{L} and V_{H} (HCDR1, HCDR2, HCDR3, and LCDR1, LCDR2, LCDR3). The heavy chain of the antibody Fab fragment is truncated after the first heavy chain domain of IgG1 (SEQ ID NO: 39), and comprises the following amino acid sequence.

(SEQ ID NO: 39)
QVQLVQSGAELKKPGASVKVSCKSSGYHFTSYWMHWVKQAPGQGL
EWIGVIHPNSGSINYNEKFESRVTITVDKSTSTAYMELSSLRSED
TAVYYCAGERDSTEVLPMDYWGQGTTVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD
KTHT

The complementarity determining regions (CDRs) of SEQ ID NO:1 are depicted in bolded and underlined text.

The light chain domain of the antibody Fab fragment comprises the following amino acid sequence (SEQ ID NO: 40):

(SEQ ID NO: 40)
DVQITQSPSSLSASLGERATINCRASKSINKYLAWYQQKPGKAPK
LLIYSGSTLQSGIPARFSGSGSGTDFTLTISSLEPEDFAMYYCQQ
HNEYPLTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC

The complementarity determining regions (CDRs) of SEQ ID NO:2 are depicted in bolded and underlined text.

### Anti-Complement C1s Antibodies

Suitable inhibitors include an antibody that binds complement C1s protein (i.e., an anti-complement C1s antibody, also referred to herein as an anti-C1s antibody and a C1s antibody) and a nucleic acid molecule that encodes such an antibody. Complement C1s is an attractive target as it is upstream in the complement cascade and has a narrow range of substrate specificity. Furthermore it is possible to obtain antibodies (for example, but not limited to, monoclonal antibodies) that specifically bind the activated form of CIs.

Examples of anti-C1s antibodies are disclosed in U.S. patent application Ser. No. 14/890,811, and U.S. Pat. No. 8,877,197, which are hereby incorporated by reference for the antibodies and related compositions that it discloses.

In certain aspects, disclosed herein are methods of administering an anti-Cis antibody. The antibody may be a murine, humanized, or chimeric antibody. In some embodiments, the light chain variable domain comprises HVR-L1, HVR-L2, and HVR-L3, and the heavy chain comprises HVR-H1, HVR-H2, and HVR-H3 of a murine anti-human CIs monoclonal antibody 5A1 produced by a hybridoma cell line deposited with ATCC on May 15, 2013 or progeny thereof (ATCC Accession No. PTA-120351). In other embodiments, the light chain variable domain comprises the HVR-L1, HVR-L2, and HVR-L3 and the heavy chain variable domain comprises the HVR-H1, HVR-H2, and HVR-H3 of a murine anti-human C1s monoclonal antibody 5C12 produced by a hybridoma cell line deposited with ATCC on May 15, 2013, or progeny thereof (ATCC Accession No. PTA-120352).

### Nucleic Acids, Vectors and Host Cells

Antibodies suitable for use in the methods of the present disclosure may be produced using recombinant methods and compositions, e.g., as described in U.S. Pat. No. 4,816,567. In some embodiments, isolated nucleic acids having a nucleotide sequence encoding any of the antibodies of the present disclosure are provided. Such nucleic acids may encode an amino acid sequence containing the V_{L}/C_{L} and/or an amino acid sequence containing the V_{H}/C_{H}1 of the anti-C1q, anti-C1r or anti-C1s antibody. In some embodiments, one or more vectors (e.g., expression vectors) containing such nucleic acids are provided. A host cell containing such nucleic acid may also be provided. The host cell may contain (e.g., has been transduced with): (1) a vector containing a nucleic acid that encodes an amino acid sequence containing the V_{L}/C_{L} of the antibody and an amino acid sequence containing the V_{H}/C_{H}1 of the antibody, or (2) a first vector containing a nucleic acid that encodes an amino acid sequence containing the V_{L}/C_{L} of the antibody and a second vector containing a nucleic acid that encodes an amino acid sequence containing the V_{H}/C_{H}1 of the antibody. In some embodiments, the host cell is eukaryotic, e.g., a Chinese Hamster Ovary (CHO) cell or lymphoid cell (e.g., Y0, NS0, Sp20 cell). In some embodiments, the host cell is a bacterium such as E. coli.

Methods of making an anti-C1q, anti-Cir or anti-C1s antibody are disclosed herein. The method includes culturing a host cell of the present disclosure containing a nucleic acid encoding the anti-C1q, anti-C1r or anti-C1s antibody, under conditions suitable for expression of the antibody. In some embodiments, the antibody is subsequently recovered from the host cell (or host cell culture medium).

### Antibody Screening

Candidate antibodies can be screened for the ability to modulate complement activation. Such screening may be performed using an in vitro model, a genetically altered cell or animal, or purified protein. A wide variety of assays may be used for this purpose, such as an in vitro culture system.

Candidate antibodies may also be identified using computer-based modeling, by binding assays, and the like. Various in vitro models may be used to determine whether an antibody binds to, or otherwise affects complement activity. Such candidate antibodies may be tested by contacting plasma from a healthy donor and determine complement activation (e.g., by the antigen C3c capture ELISA).

Generally, a plurality of assay mixtures are run in parallel with different antibody concentrations to obtain a differential response to the various concentrations. Typically one of these concentrations serves as a negative control, i.e., at zero concentration or below the level of detection.

### Pharmaceutical Compositions and Administration

A complement inhibitor (e.g. an antibody, antibody fragments and/or antibody derivatives) of the present disclosure may be administered in the form of pharmaceutical compositions.

Therapeutic formulations of an inhibitor (e.g., an antibody, antibody fragments and/or antibody derivatives) of the disclosure may be prepared for storage by mixing the inhibitor having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The inhibitor may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for administration may be sterile. This is readily accomplished by filtration through sterile filtration membranes.

The antibodies, antibody fragments and/or antibody derivatives and compositions of the present disclosure are typically administered by various routes, including, but not limited to, topical, parenteral, subcutaneous, intraperitoneal, intrapulmonary, intranasal, and intralesional administration. Parenteral routes of administration include intramuscular, intravenous, intra-arterial, intraperitoneal, intrathecal, or subcutaneous administration.

Pharmaceutical compositions may also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation may include other carriers, adjuvants, or non-toxic, nontherapeutic, non-immunogenic stabilizers, excipients and the like. The compositions may also include additional substances to approximate physiological conditions, such as pH adjusting and buffering agents, toxicity adjusting agents, wetting agents and detergents.

The composition may also include any of a variety of stabilizing agents, such as an antioxidant for example. When the pharmaceutical composition includes a polypeptide, the polypeptide may be complexed with various well-known compounds that enhance the in vivo stability of the polypeptide, or otherwise enhance its pharmacological properties (e.g., increase the half-life of the polypeptide, reduce its toxicity, enhance other pharmacokinetic and/or pharmacodynamic characteristics, or enhance solubility or uptake).

Toxicity and therapeutic efficacy of the active ingredient may be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it may be expressed as the ratio LD50/ED50. Compounds that exhibit large therapeutic indices are preferred.

The pharmaceutical compositions described herein may be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

Formulations suitable for parenteral administration include aqueous and non-aqueous, isotonic sterile injection solutions, which may contain antioxidants, buffers, bacteriostats, and solutes that render the formulation isotonic with the blood of the intended recipient, and aqueous and non-aqueous sterile suspensions that may include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for parenteral use are usually sterile. To the extent that a given compound must be synthesized prior to use, the resulting product is typically substantially free of any potentially toxic agents, particularly any endotoxins, which may be present during the synthesis or purification process. Compositions for parental administration are also typically substantially isotonic and made under GMP conditions.

The effective amount of a therapeutic composition given to a particular patient may depend on a variety of factors, several of which may be different from patient to patient. A competent clinician will be able to determine an effective amount of a therapeutic agent to administer to a patient. Dosage of the agent will depend on the treatment, route of administration, the nature of the therapeutics, sensitivity of the patient to the therapeutics, etc. Utilizing LD50 animal data, and other information, a clinician may determine the maximum safe dose for an individual, depending on the route of administration. Utilizing ordinary skill, the competent clinician will be able to optimize the dosage of a particular therapeutic composition in the course of routine clinical trials. The compositions may be administered to the subject in a series of more than one administration. For therapeutic compositions, regular periodic administration will sometimes be required, or may be desirable. Therapeutic regimens will vary with the agent; for example, some agents may be taken for extended periods of time on a daily or semi-daily basis, while more selective agents may be administered for more defined time courses, e.g., one, two three or more days, one or more weeks, one or more months, etc., taken daily, semi-daily, semi-weekly, weekly, etc.

### Methods of Treatment

The present disclosure is generally directed to methods of preventing, reducing risk of developing, slowing or blocking progression of, or treating Duchenne muscular dystrophy, Becker muscular dystrophy, Limb-Girdle Muscular Dystrophies (LGMD) (including Sarcoglycanopathies, Dystroglycanopathies and Dysferlinopathies), Collagen Type VI-Related Disorders (including Bethlem myopathy and Ullrich congenital muscular dystrophy (UCMD)), Congenital Muscular Dystrophies (CMD) and Congenital Myopathies, and Distal Muscular Dystrophies/Myopathies (including Miyoshi myopathies). The method comprises administering to a subject an inhibitor of the classical complement pathway, such as a C1 complex inhibitor, C1q inhibitor, a C1s inhibitor, or a C1r inhibitor. The inhibitor may be an antibody, a peptide, a protein, a nucleic acid, a small molecule, a gene editing agent, a base editing agent, or an epigenetic editing agent. The nucleic acid may be an antisense oligonucleotide, a miRNA, a miRNA inhibitor, an mRNA, an aptamer, or an antisense nucleic acid. The inhibitor may refer to a compound having the ability to inhibit a biological function of a target biomolecule whether by decreasing the activity or expression of the target biomolecule. Such methods include administering to a subject a C1q inhibitor. Numerous embodiments are further provided that can be applied to any aspect of the present invention described herein. For example, in some embodiments, the C1q inhibitor is an antibody, an aptamer, an antisense nucleic acid or a gene editing agent. In some embodiments, the inhibitor is an anti-C1q antibody. The anti-C1q antibody may inhibit the interaction between C1q and an autoantibody or between C1q and C1r, or between C1q and C1s, or may promote clearance of C1q from circulation or a tissue.

It is contemplated that compositions may be obtained and used under the guidance of a physician for in vivo use. The dosage of the therapeutic formulation may vary widely, depending upon the nature of the disease, the frequency of administration, the manner of administration, the clearance of the agent from the host, and the like.

### EXAMPLES

### Example 1: Materials andMethods

Unless otherwise noted, the methods and materials described in this Example 1 were used in the Examples described herein.

### Mice

Animals were maintained with access to food and water ad libitum and kept at a constant temperature (19-22° C.) on a 12:12 h light/dark cycle. C57BL/6J mice (Charles River Laboratories) were used as wild type (WT) animals. B6Ros.Cg-D^{mdmdx-4Cv/J} mice (Charles River Laboratories, herein referred to as md^{4Cv}) were used as MDX mice. For experiments with anti-C1q blocking antibody (murine antibody), Pax7-CreER^{tm} males were crossed to R26R^{YFP/YFP} females (The Jackson Laboratory) to obtain Pax7Cre^{ER/WT}; R26R^{YFP/WT} males. These breeders were crossed to mdx^{4Cv/4Cv} female to obtain the male experimental animals (Pax7Cre^{ER/WT}; R26R^{YFP/WT}; mdx^{4Cv}). Tamoxifen (T5648 Sigma) was dissolved at 50 mg/ml in 92.5% corn oil/7.5% ethanol, and 2.5 mg was administrated intraperitoneally to 54-days-old experimental mice every day for 8 days. For Lyz2Cre^{+/-}; C1qa^{FL/FL}; mdx^{4Cv} generation, C1qa^{FL/FL} (The Jackson Laboratory) males were crossed to C1qa^{WT/WT}; mdx^{4Cv/4Cv} females to obtain C1qa^{FL/WT}; mdx^{4Cv} males. These males were crossed to C1qa^{WT/WT}. mdx^{4Cv/4Cv} females to obtain C1qa^{FL/WT}; mdx^{4Cv/4Cv} females, that were crossed to C1qa^{FL/WT}; mdx^{4Cv} males to obtain C1qa^{FL/FL}; mdx^{4Cv/4Cv} females. Lyz2Cre^{+/+} (The Jackson Laboratory) males were crossed to Lyz2Cre^{-/-}; mdx^{4Cv/4Cv} females to obtain Lyz2Cre^{+/-}; mdx^{4Cv} males. These breeders were crossed to C1qa^{FL/FL}; mdx^{4Cv/4Cv} females to obtain Lyz2Cre^{+/-}; C1qa^{FL/WT}; mdx^{4Cv} males. Finally, C1qa^{FL/FL}; mdx^{4Cv/4Cv} females were crossed to Lyz2Cre^{+/-}; C1qa^{FL/WT}; mdx^{4Cv} males to obtain the male experimental animals (Lyz2Cre^{+/-}; C1qa^{FL/FL}; mdx^{4Cv}, hereinafter referred to as C1qa^{KO};mdx^{4Cv} mice). Genotyping was performed with primers listed in Table 1.

TABLE 1
Primer list for genotyping PCR

| Gene | Forward Primer | Reverse Primer | T_{Ann} |
|---|---|---|---|
| C1qa | tgcatcctg | gaaagtgcttaaag | 60° C. |
| | ccatctcct | aaaccactg | |
| | (SEQ ID NO: 41) | (SEQ ID NO: 42) | |
| Cre | gcatttctg | cccggcaaaa | 53.7° C. |
| | gggattgctta | caggtagtta | |
| | (SEQ ID NO: 43) | (SEQ ID NO: 44) | |
| YFP | aagttcatctg | tccttgaagaaga | 53.7° C. |
| | caccaccg | tggtgcg | |
| | (SEQ ID NO: 45) | (SEQ ID NO: 46) | |

### Behavioral Test

Before behavioral tests, mice were handled on a regular basis from the same operator to limit stress levels. Tests were consistently performed at the same time of the day during the mice dark phase hours. All the behavioral tests were performed in blind.

### Four Limbs Hanging Test

Four limbs hanging test protocol was adapted from the Treat-NDM Neuromuscular Network SOP DMD_M2.2.1.005. Prior to the test, mice were weighed to allow normalization for body weight. A 40 cm×30 cm metallic grid was placed 40 cm above a desk with sufficient bedding (i.e., papers and wood chips) to ensure a soft landing. Mice were placed on the grid and were allowed to acclimate to this environment for 3 to 5 seconds before the grid was inverted and held at 40 cm of height. If mice fell off the grid before the fixed time limit (900 secs), they were immediately given two more tries. The total hanging time was recorded. The test was repeated every other day for three times.

### Two Limbs Grip Test

The two Limbs Grip Strength test protocol was adapted from the Treat-NMD Neuromuscular Network SOP DMD_M2.2.001. Prior to the test, mice were weighed to allow normalization for body weight. Mice were allowed to attach only with their forelimbs to a metallic trapeze connected to a force transducer and an electronic unit (47200 —Grip-Strength Meter—Ugo Basile). Mice were gently pulled by their tail. The maximal grip strength and the total time of the test (i.e., total time in which mice were attached to the trapeze) were recorded four times and the average values of these measurements were calculated. The test was repeated six times (in C1qaKO e Cntr mice: three tests on three consecutive days, then after five days three further tests on alternate days) or three times (in WT mice on alternate days).

### Open Field Test

Open field test protocol was adapted from the Treat-NDM Neuromuscular Network SOP DMD M2.2.1.002. Immediately after the four limb hanging test, mice were placed into a 40 cm×40 cm arena and allowed to move freely and explore for 5 or 6 minutes. The test was recorded and movement data were analyzed using ANY-maze software or EthoVision XT software. The test was repeated every other day for three times.

### Rotarod Test

The rotarod test was performed using LE8205 Panlab Harvard Apparatus. Prior to the test, mice were weighed to allow normalization for body weight. The speed of the rotating rod was set to a constant value of 6 rpm. After one hour, the speed was increased to a constant value of 12 rpm. If mice fell off the rod, they were immediately given three more tries. The total walking time was recorded. The test was repeated for six days (three tests on three consecutive days, then after five days three further tests on alternate days).

### Plasma and Sera Collection and Preparation

Mice were heated under a lamp for few minutes to increase the blood flow. The area of the submandibular vein was pierced with the tip of a needle and the blood flow from the cheek was collected in a tube. Blood samples were incubated at room temperature for 1 hour, centrifuged at 2.000 g for 10 minutes at 4° C. The supernatant (serum) was collected. Alternatively, blood was collected into EDTA-treated tubes. Samples were centrifuged at 2.000 g for 10 minutes at 4° C. and the supernatant (plasma) was collected.

### Muscle Single Cells Isolation

Hindlimb muscles (i.e., gastrocnemius, EDL, tibialis anterior and quadriceps) from wild type and dystrophic mice were dissected and processed as previously described to obtain mononucleated cells. Muscles were washed in Wash Medium (Ham's F-10 supplemented with 10% FBS, 1% L-glutamine and 1% penicillin-streptomycin), added to Muscle Dissociation Buffer (700-800 U/ml collagenase II (Worthington Biochemical Corporation) prepared in Ham's F-10 supplemented with 1% L-Glutamine and 1% penicillin-streptomycin) (16 ml/hindlimb, 8 ml/diaphragm), minced with scissor, incubated in a 37° C. water bath with agitation (70 rpm) for 40 minutes. After incubation samples were centrifuged at 500 g for 5 minutes, dispase (11 U/ml, GIBCO) and collagenase II (235 U/mg) (1.5 ml/hindlimb, 0.5 ml/diaphragm) were added to induce enzymatic digestion and the pellet was resuspended and triturated with a 10-ml serological pipette. Samples were incubated in a 37° C. water bath with agitation (70 rpm) for 20 minutes, then passed through 18- and 19-gauge needles by using a 10 ml syringe to allow mechanical dissociation of the cell suspension into single cells. Samples were centrifuged at 500 g for 10 minutes at 4° C., the pellet was resuspended and filtered through a 40 m nylon cell strainer (Euroclone), centrifuged (500 g, 10 minutes at 4° C.), then resuspended in Wash Medium. Cells were incubated on a rotating wheel (10 rpm) for 45 minutes at 4° C. with primary antibodies to mark macrophages, fibro-adipogenic progenitors and satellite cells. A list of primary antibodies used is enclosed in Table 2. Samples were washed, APC Streptavidin (1:100, BioLegend) was added and samples were incubated on a rotating wheel (10 rpm) for 20 minutes at 4° C., then washed and resuspended in a sorting buffer (Wash Medium+(PBS with 1.5 mM EDTA, 2% BSA, 1% L-glutamine and 1% penicillin-streptomycin), 1:1 ratio). Samples were finally filtered through cell strainer cap tubes (Thermo Fisher Scientific).

TABLE 2
Primary antibodies used to FACS isolate muscle cells.

| Antibody | Company | Dilution |
|---|---|---|
| APC/Fire 750 anti-mouse CD45 | BioLegend (103154) | 1:100 |
| APC/Fire 750 anti-mouse CD31 | BioLegend (102434) | 1:100 |
| FITC anti-mouse F4/80 | BioLegend (123107) | 1:100 |
| Brillant Violet 421 | BioLegend (108127) | 1:100 |
| anti-mouse Ly-6A/E (Scal) | | |
| Biotin anti-mouse CD016 (Vcam) | BioLegend (105704) | 1:100 |

FACS Aria™ II cell sorter (BD Biosciences) was used to separate cell populations. Physical parameters as forward scatter (FSC) and side scatter (SSC) were used to exclude cell clumps, debris, dead cells and to isolate single cells. Satellite cells were purified by negative selection with anti-CD31, anti-CD45, anti-Scal antibodies and positive selection with anti-Vcam antibody, fibro-adipogenic progenitors were purified by negative selection with anti-CD31 and anti-CD45 antibodies and positive selection with anti-Scal antibody, macrophages were purified by positive selection with anti-CD45 and anti-F4/80 antibodies. In samples collected from Pax7CreER/WT; R26RYFP/WT; mdx^{4Cv} mice satellite cells were purified by isolating the YFP+ve cell population. After sorting, cells were processed for RNA extraction and RT-PCR.

### Real-Time PCR

Prior to RNA extraction, muscles were dissected and snap frozen. Snap frozen muscles were homogenized using a mortar and pestle on dry ice under liquid nitrogen. Total RNA was extracted from muscles and from FACS isolated cells using TRIzol@Reagent (Invitrogen) according to manufacturer's instructions. RNA was quantified using NanoDrop spectrophotometer and reverse transcribed using High Capacity cDNA Reverse Transcription Kit (Thermo Fisher Scientific) according to manufacturer's instructions. Gene expression was measured by quantitative RT-PCR using SYBR Green Master Mix (Thermo Fisher Scientific) and a C1000 Touch thermocycler—CFX96 Real Time System (Biorad). The thermo protocol used for RT-PCR is indicated in Table 3. Primers spanning exon-exon junctions were used (Table 4). The level of each transcript was measured using mouse HPRT (hypoxanthine-guanine phosphoribosyltransferase) mRNA levels as normalizer.

TABLE 3
RT-PCR thermo protocol

| Step | Temperature (° C.) | Time (min) |
|---|---|---|
| 1 | 50.0 | 2.00 |
| 2 | 95.0 | 5.00 |
| 3 | 95.0 | 0.15 |
| 4 | 60.0 | 1.00 |
| 5 | 72.0 | 0.30 (Plate read) |
| 6 | Go to step 3 (39×) | |
| 7 | 65.0 − 95.0 | 0.05 − 0.5 (Plate read) |

TABLE 4
Primer list for SYBER Green RT-PCR

| | | | | Span an |
|---|---|---|---|---|
| | | | | exon- |
| | | | | exon |
| Gene | Forward Primer | Reverse Primer | T_{Ann} | junction? |
| mAxin2 | cagagggacaggaaccactc | tgccagtttctttggctctt (SEQ ID | 60° C. | Yes |
| | (SEQ ID NO: 73) | NO: 48) | | |
| mC1qa | tctcagccattcggcagaac | tggttggtgaggaccttgtca (SEQ ID | 60° C. | No |
| | (SEQ ID NO: 49) | NO: 50) | | |
| mCol1a1 | tccggctcctgctcctctta (SEQ | gtatgcagctgacttcagggatgt (SEQ | 60° C. | Yes |
| | ID NO: 51) | ID NO: 52) | | |
| mCol3a1 | gcccacagccttctacac (SEQ | ccagggtcaccatttctc (SEQ ID | 60° C. | Yes |
| | ID NO: 53) | NO: 54) | | |
| mFn1 | tgcctcgggaatggaaag (SEQ | atggtaggtcttcccatcgtcata (SEQ | 60° C. | Yes |
| | ID NO: 55) | ID NO: 56) | | |
| mLGR5 | tcgccttccccaggtcccttc | gccgtggtccacaccccgat (SEQ ID | 60° C. | Yes |
| | (SEQ ID NO: 57) | NO: 58) | | |
| mTGFβ2 | cgagcggagcgacgaggagt | tgggcgggatggcattttcgg (SEQ ID | 60° C. | Yes |
| | (SEQ ID NO: 59) | NO: 60) | | |
| mHPRT | aactggaaagaatgtcttgattgt | gaatttcaaatccaacaaagtctgg (SEQ | 60° C. | Yes |
| | (SEQ ID NO: 61) | ID NO: 62) | | |
| mC1qb | gggaatccactgctgtccggc | ctcagcctcaggggcttcctgt (SEQ ID | 60° C. | Yes |
| | (SEQ ID NO: 63) | NO: 64) | | |
| mC1qc | agagccaggaatcccagccgtcc | gcatgccaggctcgccctt (SEQ ID | 60° C. | No |
| | (SEQ ID NO: 65) | NO: 66) | | |
| mC1r | aaccatattacaagatgctgacca | ccttgggctgtgcaggta (SEQ ID NO: | 60° C. | Yes |
| | (SEQ ID NO: 67) | 68) | | |
| mC1s | gaccagaggcaggagaggaggc | gctcagtgtcaccttcaggagc (SEQ ID | 60° C. | Yes |
| | (SEQ ID NO: 69) | NO: 70) | | |
| mHPRT | tcagaccgctttttgccgcga (SEQ | atcgctaatcacgacgctgggac (SEQ ID | 60° C. | No |
| | ID NO: 71) | NO: 72) | | |

### Creatine Kinase Test

Creatine kinase test was performed on serum samples collected from ˜3 months old C1qaKO;mdx^{4Cv} and Pax7^{CreER};R26R^{YFP};mdx^{4Cv} mice prior to sacrifice. The Creatine Kinase Activity Assay Kit (Colorimetric) (Abcam, 155901) was used for the analysis according to the manufacturer's instructions.

Immunofluorescence Muscle sections were processed for immunofluorescence as known in the art. Briefly, dissected muscles were fixed for 4 hours using 0.5% paraformaldehyde, then transferred to 30% sucrose overnight, frozen in optimum cutting temperature compound (OCT), and cryosectioned at 8 m. The acquisition was made with a Zeiss Axio Observer Z1 optical microscope equipped with a monochrome camera (AxioCam 503 mono D). Anti-Axin2 (ab32197, 1:20) and anti-C1q (ab11861, 1:50) were used as primary antibodies. Alexa Fluor 488/594 (Thermo Fisher Scientific) were used as secondary antibodies.

Zen 2 software (Zeiss) was used for immunofluorescence analysis. The average pixel intensity of C1q and Axin2 was measured for each biological replicate in ≥106 randomly selected regions of 1017 μm² within the regenerating areas of the muscle. The background pixel intensity measured on sections stained only with the secondary antibody was subtracted. Normal or non-normal data set distribution, Spearman (r), and Pearson coefficient (r) were determined with GraphPad Prism software.

### Statistical Analysis

Unless otherwise stated, experiments presented here were repeated at least three times. Unless otherwise stated, data are presented as mean±SEM. Statistical analysis was performed using GraphPad Prism 8. One-way ANOVA test was performed for multiple comparisons; parametric Student-t test was performed for comparison between two groups. The number of biological replicates and the use of specific tests has been reported in each figure legend. Statistical significance was expressed with p-value (p): p>0.05: ns; p≤0.05: *; p≤0.01: **; p≤0.001: *** and p≤0.0001: ****.

### Tissue Lysis

All tissues (e.g., skeletal muscles, etc.) were weighed and resuspended in 1:10 w/v of lysis buffer (BupH™ Tris Buffered Saline (Thermo Scientific 28379)+protease inhibitor cocktail (Thermo Scientific A32963)+10 mM EDTA) by homogenizing with 7 mm steel bead in Qiagen TissueLyser for 2 minutes at 30 Hz. Lysates were then spun at 17,000×g for 20 minutes. Supernatants were used for ELISA assays. Total protein was measured using the PIERCE™ BCA Protein Assay kit (ThermoFisher 23225).

### PK and Complement Assays

The levels of free anti-C1q-blocking murine antibody, free C1q, total C1q, C1s, C4, C2, C3 and activation markers C1q-C3d complex, C1s-C1inh complex and C3d were measured in plasma and tissue lysates using sandwich ELISAs. Black 96 well plates (Costar #3925) were coated with 75 μL of respective capture antibody (Table 5) in bicarbonate buffer (pH 9.4) overnight at 4 C. Next day, the plates were washed with dPBS pH 7.4 (Dulbecco's phosphate-buffered saline) and then blocked with dPBS buffer containing 3% bovine serum albumin (BSA). Standard curves were prepared with purified proteins in assay buffer (dPBS containing 0.3% BSA, 0.1% Tween20, 10 mM EDTA). Study serum/plasma samples were prepared in the assay buffer at respective dilutions. The blocking buffer was removed from the plate by tapping. Standards and samples were added at 75 μL per well in duplicates and incubated with shaking at 300 rpm at room temperature for 1 hr for PK measurements, and subsequently overnight at 4 C for all other assays. Plates were washed thrice with wash buffer (dPBS containing 0.0500 Tween20) and 75 μL of alkaline-phosphatase conjugated secondary antibodies (Table 5) were added to all wells. Plates were incubated at room temperature with shaking for 1 h. Plates were washed thrice with wash buffer and developed using 75 μL of alkaline phosphatase substrate (Life Technologies, T2214). After 20 minutes at room temperature, plates were read using a luminometer. Standards were fit using a 4PL logistic fit and concentration of unknowns determined. Analyte levels were corrected for dilution and then plotted using GraphPad Prism.

TABLE 5
Standards and antibodies used in ELISA assays

| | | | Plasma | Tissue |
|---|---|---|---|---|
| | | | dilution | dilution |
| Assay | Capture | Secondary | factor | factor |
| PK | Human C1q (Complement | Goat Anti-Mouse IgG | 300000 | 1000 |
| | Tech A100) | (JacksonImmuno 115- | | |
| | | 055-071) | | |
| C1q Free | anti-C1q (JL1, abcam | anti-C1q -AP | 15000 | 30 |
| | 71940) | (Annexon) | | |
| C1q Total | anti-C1q (JL1, abcam | anti-C1q-AP (7H8, | 1000 | |
| | 71940) | abcam 11861) | | |
| C1s | polyclonal anti-C1s | polyclonal anti-C1s-AP | 1000 | 10 |
| | (Annexon) | (Annexon) | | |
| C4 | anti-C4 (LSBio C374031) | anti-C4-AP (Invitrogen | 300 | |
| | | MA1-40047) | | |
| C2 | Polyclonal anti-C2 | Polyclonal anti-C2-AP | 100 | |
| | (Annexon) | (Annexon) | | |
| C3 | anti-C3d (Dako A063) | anti-C3-AP (MPBio | 1000 | 300 |
| | | 0855444) | | |
| C3d | anti-C3d (Dako A063) | anti-C3-AP (Novus Bio | 1000 | 30 |
| | | NB200-540) | | |
| C1q-C3d | anti-C1q (Dako A0136) | anti-C3d-AP (Annexon) | 500 | 10 |
| complex | | | | |
| C1s-Clinh | Monoclonal anti-C1s | Monoclonal anti-Clinh- | 100 | 10 |
| complex | (Annexon) | AP (Annexon) | | |

| | | |
|---|---|---|
| Albumin | abcam 210890 kit | 50000 |

### Example 2: Evaluation of Complement Levels in the Skeletal Muscles of Wild Type, Dystrophic and Old Wild Type Mice

The levels of the classical complement proteins C1q, C3 and C3d were evaluated through ELISA in the diaphragm, quadriceps and tibialis anterior of ˜1 year old wild type, ˜2 years old wild type and ˜1 year old mdx^{4Cv} mice.

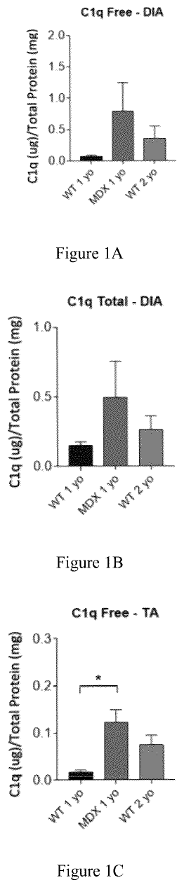

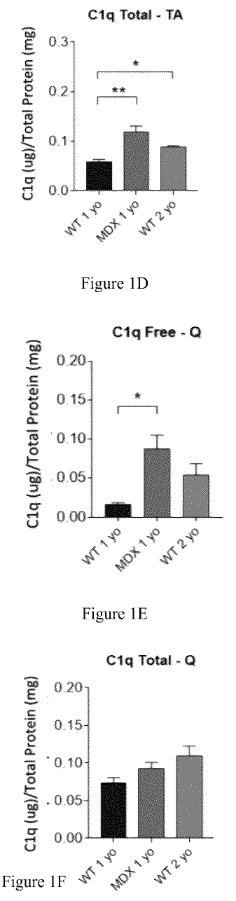

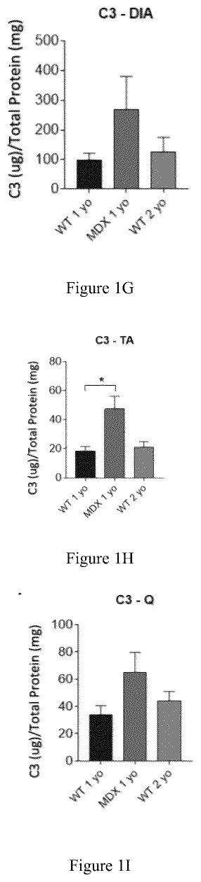

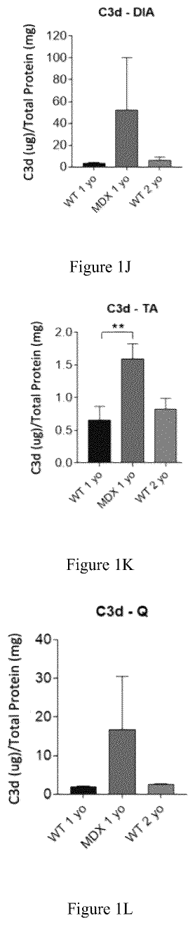

FIG. 1: Increased levels of C1q, C3 and C3d were observed in the tibialis anterior of the mdx^{4Cv} mice compared to the age matched controls (˜1 year old) and increased levels of C1q were observed in the tibialis anterior of ˜2 years old mice compared to ˜1 year old wild type. Trends suggesting increased levels of C1q, C3 and C3d were observed in the diaphragm of the mdx^{4Cv} mice compared to the age matched controls (˜1 year old). C1q levels were increased in the quadriceps of the mdx^{4Cv} mice compared to the age matched controls (˜1 year old) and both C3 and C3d levels exhibit a similar trend. Finally, a trend suggesting an increase of the C1q and C3 levels was observed in the quadriceps of ˜2 years old mice compared to ˜1 year old wild type.

The levels of the classical complement proteins C1q, C1s, C3, C3d and C4 were evaluated through ELISA in diaphragms, quadriceps and tibialis anterior of ˜1 month, ˜3 months and ˜1 year old wild type and mdx^{4Cv}. Importantly, mice were perfused with PBS prior to muscle dissection and ELISA with the aim of restricting the evaluation to the muscles and excluding the serum protein components from the analysis. C1q, C1s, C3, C3d and C4 were expressed in all the analyzed wild type and mdx^{4Cv} muscles.

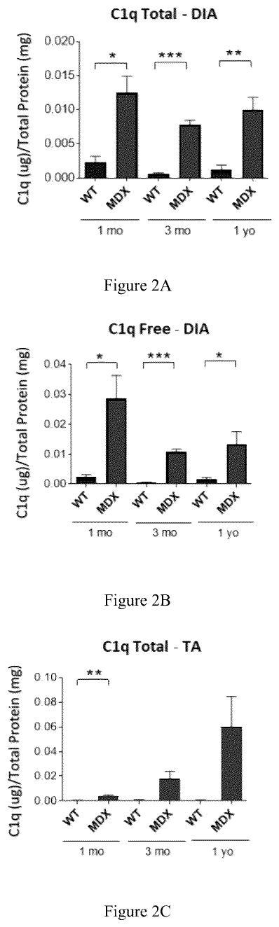

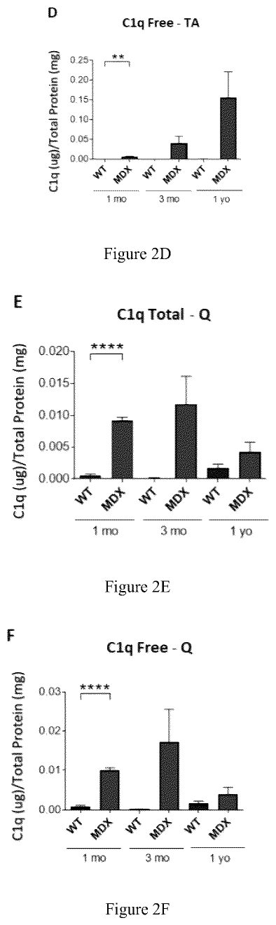

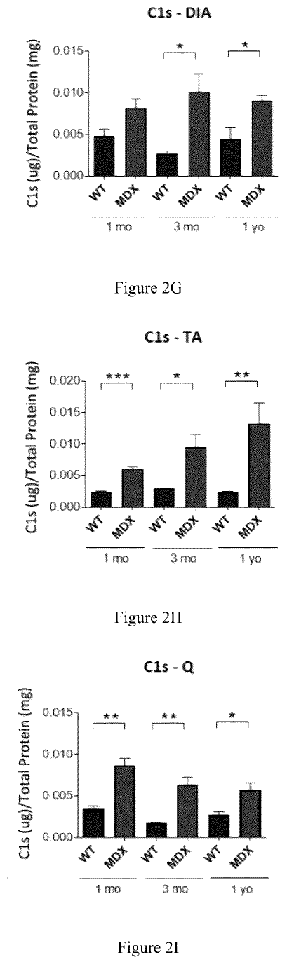

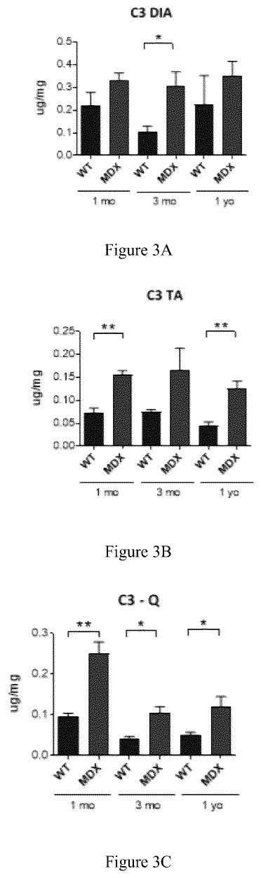

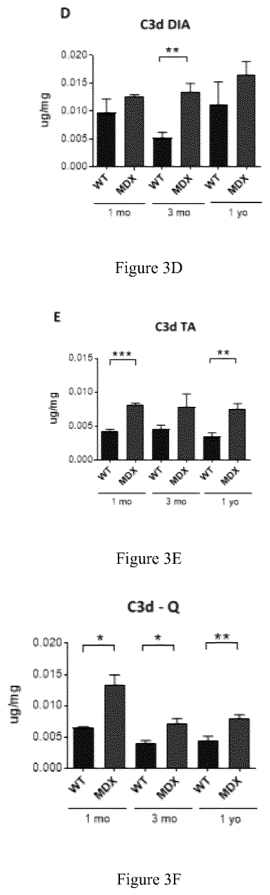

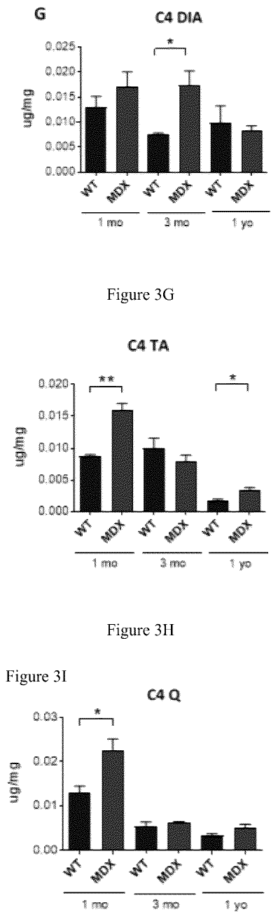

FIGS. 2 and 3: C1q, C1s, C3 and C3d protein expression was higher in the ˜1 month, ˜3 months and ˜1 year old dystrophic muscles compared to the wild type in the diaphragm, quadriceps and tibialis anterior, reaching statistical significance in most cases. C4 protein expression was higher in the dystrophic tibialis anterior and quadriceps of ˜1 month old mice compared to the wild type, in the diaphragm of ˜3 months old dystrophic mice compared to the wild type and in the tibialis anterior of ˜1 year old dystrophic mice compared to the wild type.

### Example 3: Behavioral Tests in Wild Type and Dystrophic Mice

Behavioral tests were performed on the same wild type and dystrophic ˜1 month, ˜3 months and ˜1 year old mice analyzed for complement protein expression (FIGS. 2 and 3). The following functional parameters were evaluated: 1) the maximal hanging time before exhaustion was assessed through the four-limb hanging wire test; 2) the total distance travelled, the mean speed and the percentage of mobile time were assessed through the open field test.

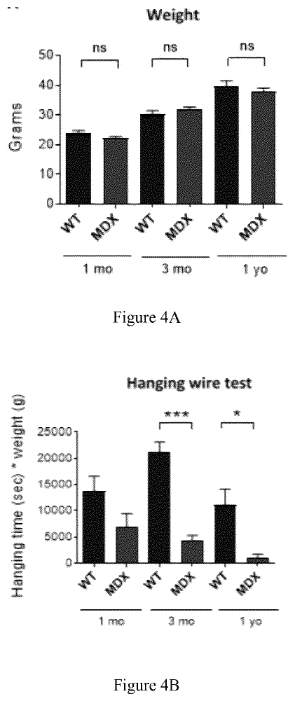

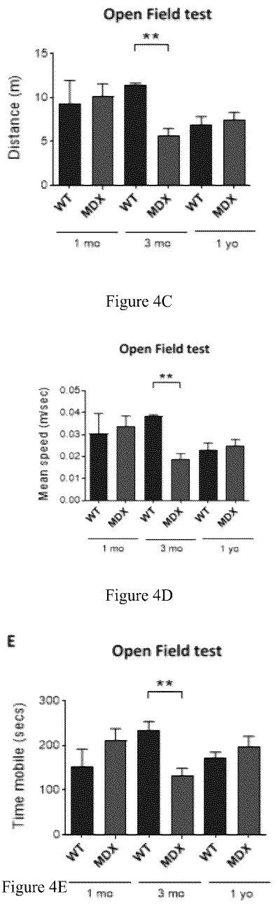

FIG. 4: The wild type and dystrophic mice exhibited a differential muscle resistance during behavioral tests performed before muscle dissection and ELISA, in keeping with the reported motor deficit occurring in the mdx mice compared to healthy controls. Dystrophic mice were less resistant during the four limbs hanging wire test compared to the age-matched wild type at ˜3 months and ˜1 year old. In the open field test, ˜3 months old dystrophic mice walked a shorted distance at a lower speed and spent more immobile time compared to the wild type, whereas no differences were observed among ˜1 months and ˜1 year old dystrophic mice compared to the age-matched wild type.

### Example 4: Evaluation of the Efficacy of Anti-C1q-Blocking Antibody In Vivo

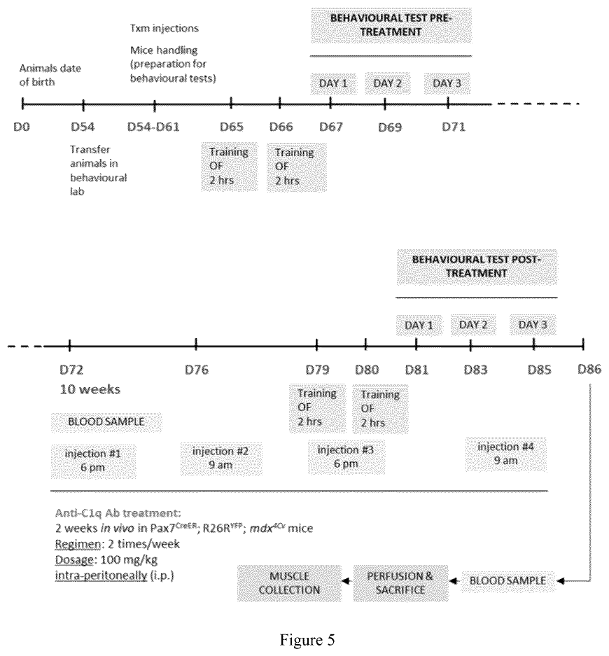

Pax7^{CreER};R26R^{YFP};mdx^{4Cv} mice were treated with the anti-C1q blocking antibody or with the control antibody. Behavioral tests were performed before and after the treatment in order to evaluate functional parameters (i.e., maximum time before exhaustion and locomotor activity). Blood samples were collected before and after the treatment in order to evaluate complement levels and creatine kinase levels. Tissues (i.e., hindlimb muscles, diaphragm, liver and heart) were collected at sacrifice in order to perform complement levels and gene expression analysis. The experimental plan followed for the anti-C1q blocking antibody treatment is enclosed in FIG. 5.

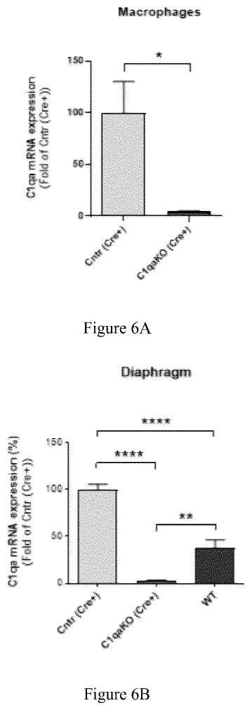

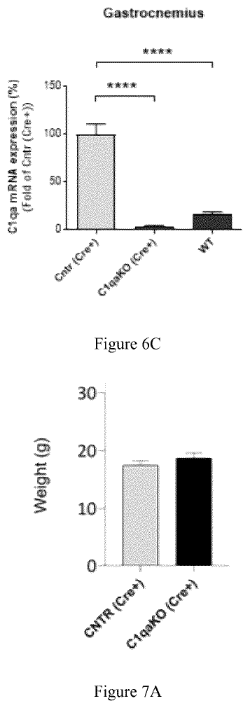

Behavioral tests and samples' (i.e., blood and tissues) collection were performed also on the C1qa^{KO};mdx^{4Cv} mice in which the expression of C1qa is conditionally ablated in the myeloid cell line. The C1qa^{KO};mdx^{4Cv} mouse validation is enclosed in FIG. 6.

### Example 5: Behavioral Test in C1qa^{KO}; mdx^{4Cv} Mice and in Dystrophic Mice Treated with Anti-C1q Blocking Antibody

Behavioral tests were performed on C1qa^{KO};mdx^{4Cv} mice at ˜1 month, ˜2 months and ˜3 months of age in order to evaluate the maximal hanging time before exhaustion (through the four limb hanging wire test), the total distance travelled, the mean speed and the percentage of mobile time (through the open field test).

The same behavioral test were performed on the Pax7^{CreER};R26R^{YFP};mdx^{4Cv} mice treated with anti-C1q blocking antibody or with the control antibody before and after the treatment.

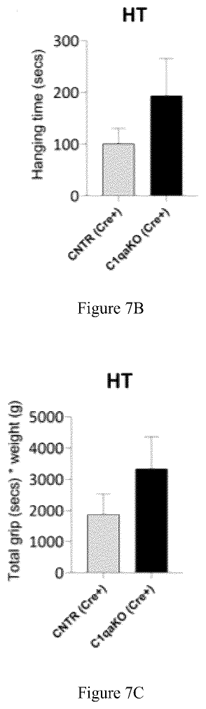

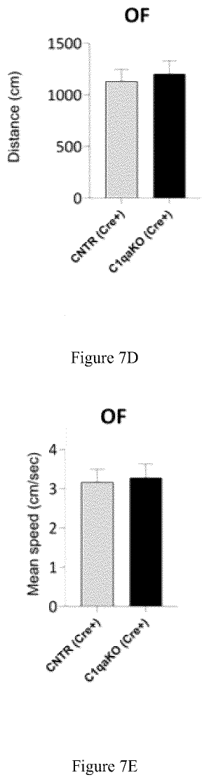

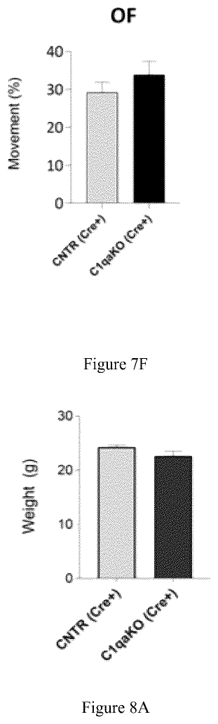

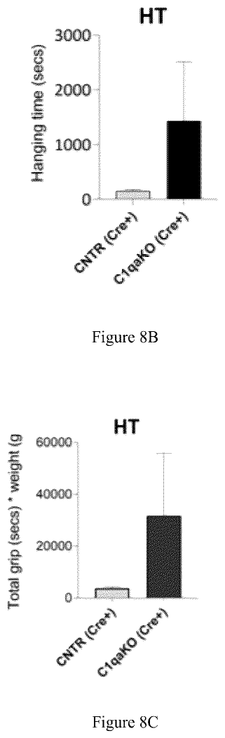

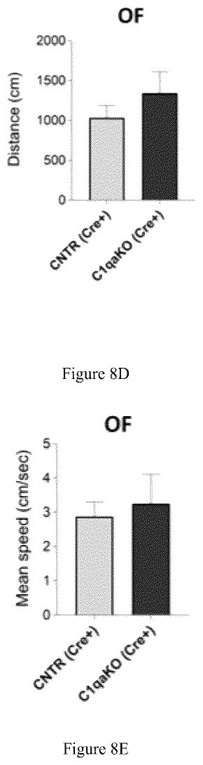

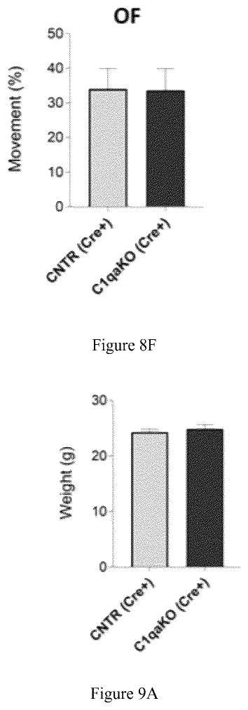

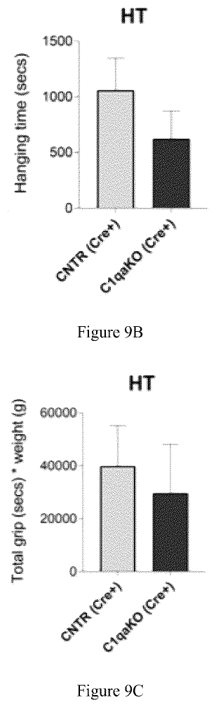

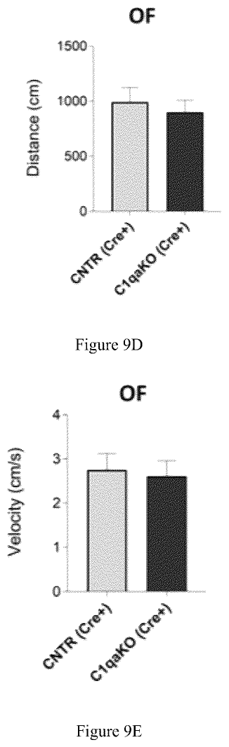

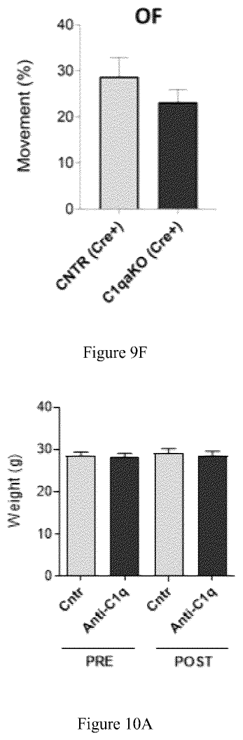

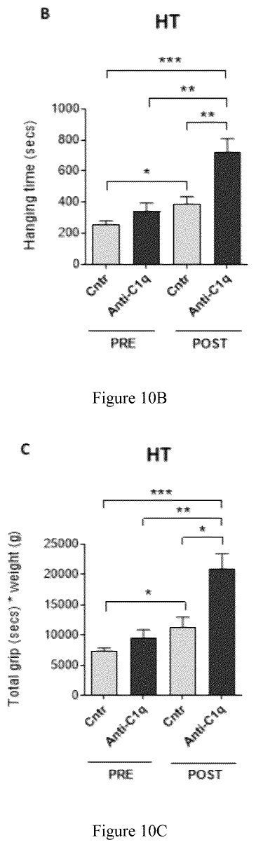

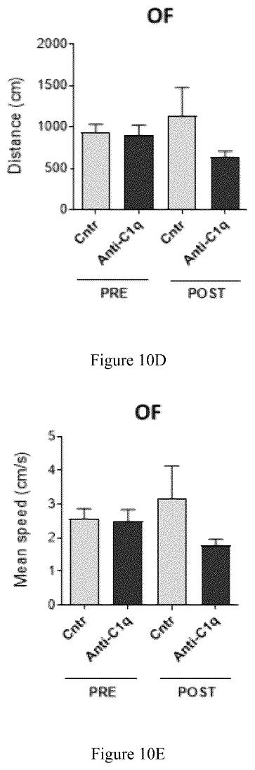

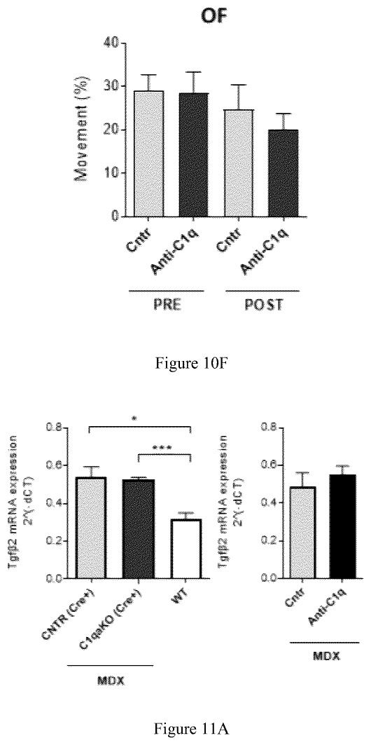

FIGS. 7-10: At ˜1 month of age (i.e., first day of the test at day 31^{st}-36^{th} after birth) we observed a trend suggesting an improved physical resistance in the C1qa^{KO};mdx^{4Cv} mice compared to the controls in the four limb hanging wire test. The same trend was observed at ˜2 months of age (i.e., first day of the test at day 67^{th}-69^{th} after birth), but it was no longer observed when mice were tested at ˜3 months of age (i.e., first day of test at day 79^{th}-81^{st} after birth). No differential locomotor activity was observed in the C1qa^{KO};mdx^{4Cv} mice compared to the controls in the open field test at any of the tested time points (i.e., ˜1 month, ˜2 months and ˜3 months).

Dystrophic mice treated with the anti-C1q blocking antibody resulted physically more resistant compared to the mice treated with the control antibody in the four-limb hanging wire test. No differences were observed in the open field test parameters (i.e., total distance, mean speed and percentage of mobile time) in the dystrophic mice treated with anti-C1q blocking antibody compared to the controls. To note, the behavioral tests were performed in these animals before the treatment and after the treatment at ˜2.5 months of age (i.e., first day of the test at day 67^{th} after birth) and ˜3 months of age (i.e., first day of test at day 81^{st} after birth), respectively (FIG. 5).

### Example 6: Gene Expression Evaluation of Canonical Wnt Target Genes and Fibrogenic Genes in C1qa^{KO}; Mdx^{4Cv} Mice and in Dystrophic Mice Treated with Anti-C1q Blocking Antibody

We performed a gene expression analysis in muscles and in single cells isolated from the C1qa^{KO};mdx^{4Cv} mice and from the Pax7^{CreER};R26R^{YFP};mdx^{4Cv} mice treated with anti-C1q blocking antibody in order to evaluate whether C1q depletion led to a reduction of the expression of the canonical Wnt pathways and/or to reduced fibrosis levels. The mRNA levels of canonical Wnt target genes (i.e., Tgfβ2, Lgr5 and Axin2) and fibrogenic genes (i.e., collagen1a1, collagen3a1 and fibronectin) were evaluated in the muscles (i.e., gastrocnemius and diaphragm) and in the isolated single cells (i.e., fibro/adipogenic progenitors and satellite cells) of C1qa^{KO};mdx^{4Cv} mice and Pax7^{CreER};R26R^{YFP};mdx^{4Cv} mice treated with anti-C1q blocking antibody.

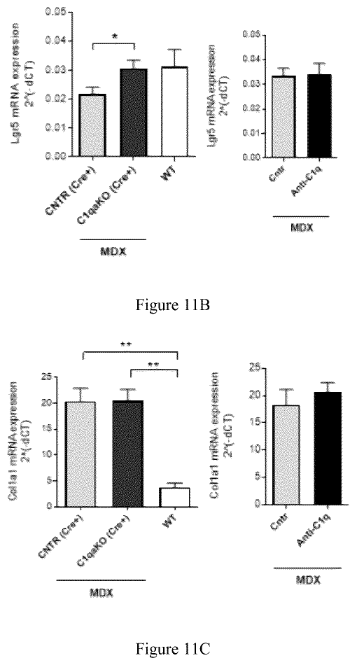

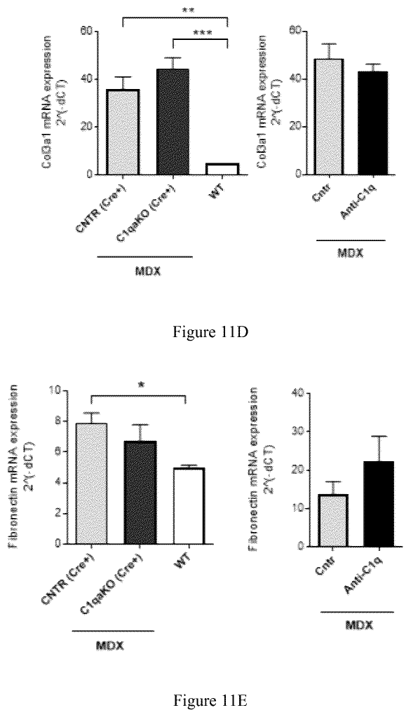

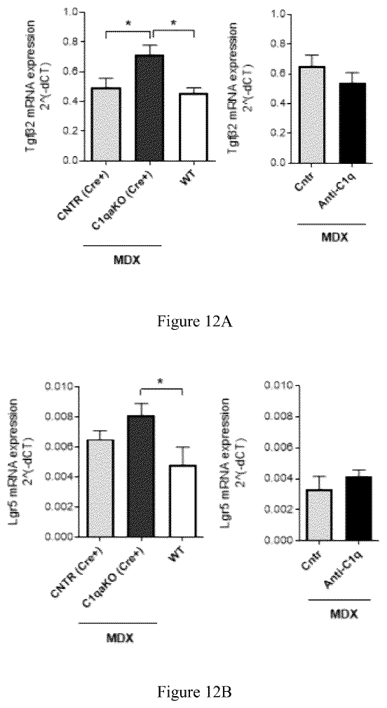

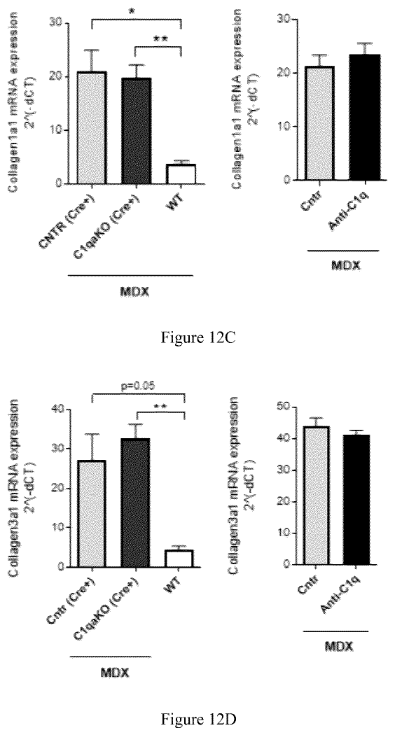

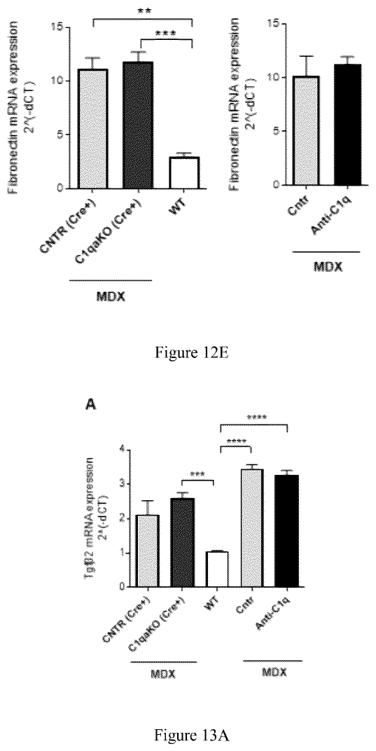

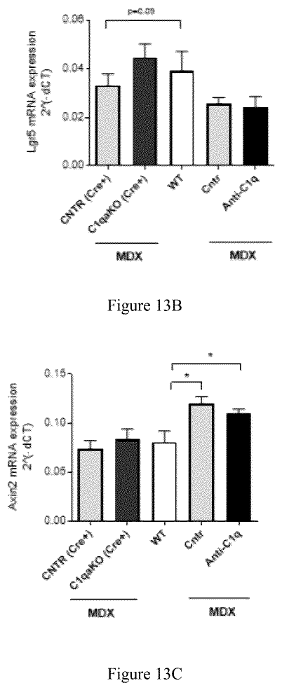

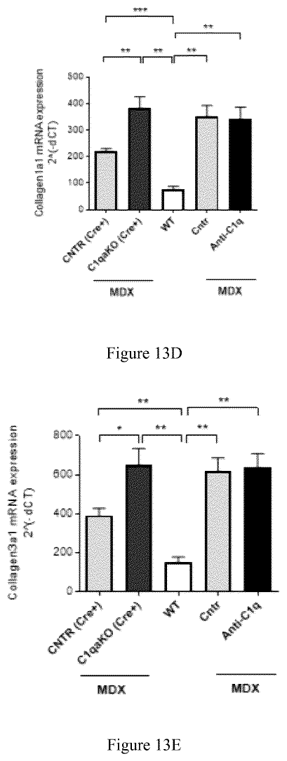

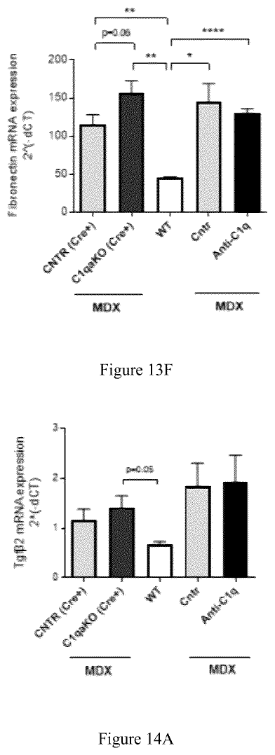

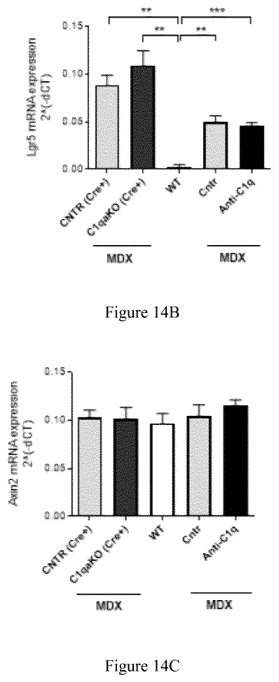

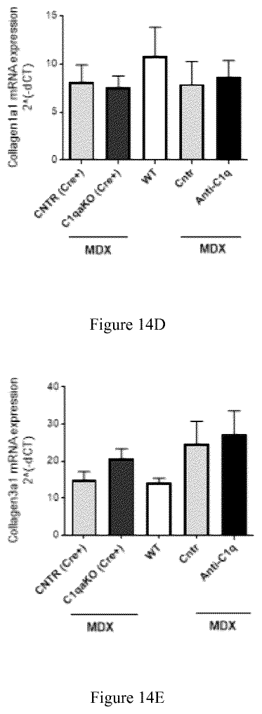

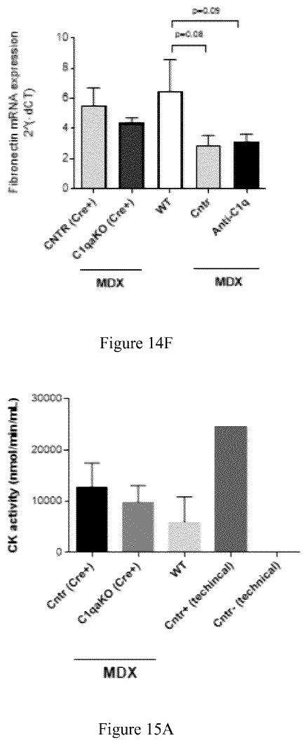

FIGS. 11-14: Overall, we did not observe a reduced expression of either the Wnt target genes (i.e., Tgfβ2, Lgr5 and Axin2) or the fibrogenic genes (i.e., collagen1a1, collagen3a1 and fibronectin) in the C1qa^{KO};mdx^{4Cv} muscles and single cells compared to the controls.

We also did not observe a reduced expression of either the Wnt target genes (i.e., Tgfβ2, Lgr5 and Axin2) or the fibrogenic genes (i.e., collagen1a1, collagen3a1 and fibronectin) in the muscles and in the single cells of the Pax7^{CreER};R26R^{YFP};mdc^{4Cv} mice treated with anti-C1q blocking antibody compared to the controls.

FIG. 27 shows representative immunofluorescence of gastrocnemius of ˜1 year-old mdx^{4Cv} stained with anti-C1q, anti-Axin2 antibodies, and Hoechst. Scale bar (top images). The positive correlation between C1q and Axin2 intensity values in each region is shown.

### Example 7: Creatine Kinase Test on Serum from C1Qa^{KO};Mdx^{4Cv} Mice and from Dystrophic Mice Treated with Anti-C1q Blocking Antibody

Serum creatine kinase (CK) levels are commonly used as an indicator of muscle damage in dystrophic mice and as diagnostic biomarker for DMD. In order to evaluate whether C1q depletion led to a reduction of the CK levels, we measured the CK activity in C1qa^{KO};mdx^{4Cv} mice and in Pax7^{CreER};R26R^{YFP};mdx^{4Cv} mice treated with anti-C1q blocking antibody. The CK test was performed in both cases using serum samples collected at sacrifice (˜3 months old).

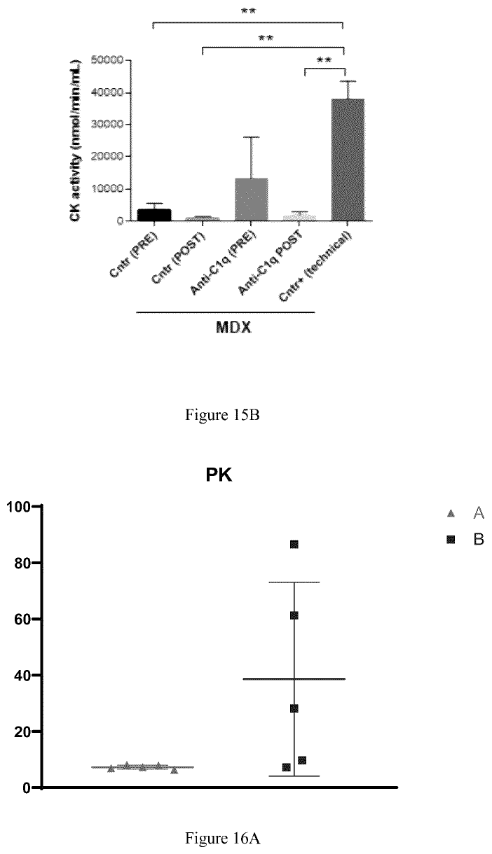

FIG. 15: We observed a trend suggesting a reduction of the CK activity in the C1qa^{KO};mdx^{4Cv} serum compared to the controls.

We observed trends suggesting a reduction of the CK activity in the Pax7^{CreER};R26R^{YFP};mdx^{4Cv} mice after the treatment with the C1q-blocking antibody and with the control antibody. However, the different CK activity levels measured in the same samples before the treatment (Cntr (PRE) and Anti-C1q (PRE) in FIG. 15 B, supposedly similar) suggest biological variability.

### Example 8: Complement Levels Evaluation in Plasma and Muscles Collected from C1qa^{KO};mdx^{4Cv} Mice and Dystrophic Mice Treated with Anti-C1q Blocking Antibody

ELISA assays were performed in order to evaluate the complement levels in plasma and in tissues collected from the C1qa^{KO};mdx^{4Cv} mice and from the dystrophic mice treated with anti-C1q blocking antibody. The levels of the classical complement proteins (i.e., C1q, C3d and C1s), the C1q-C3d immune complex (IC) and C1s-C1inhibitor complex (C1sC1inh) were evaluated in the plasma, diaphragm, gastrocnemius and liver collected from C1qa^{KO};mdx^{4Cv} mice and from the dystrophic mice treated with anti-C1q blocking antibody. In samples collected from the dystrophic mice treated with the anti-C1q blocking antibody the drug level present in the samples was also evaluated (PK). Moreover, all the aforementioned proteins' levels were evaluated in the heart samples collected from the dystrophic mice treated with the anti-C1q blocking antibody.

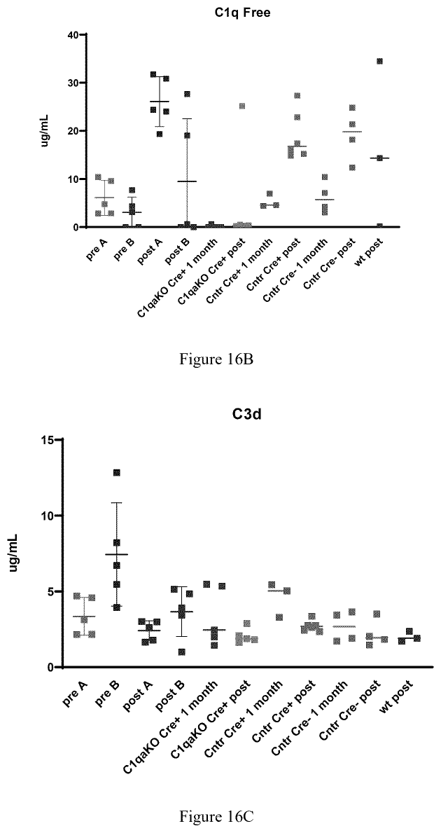

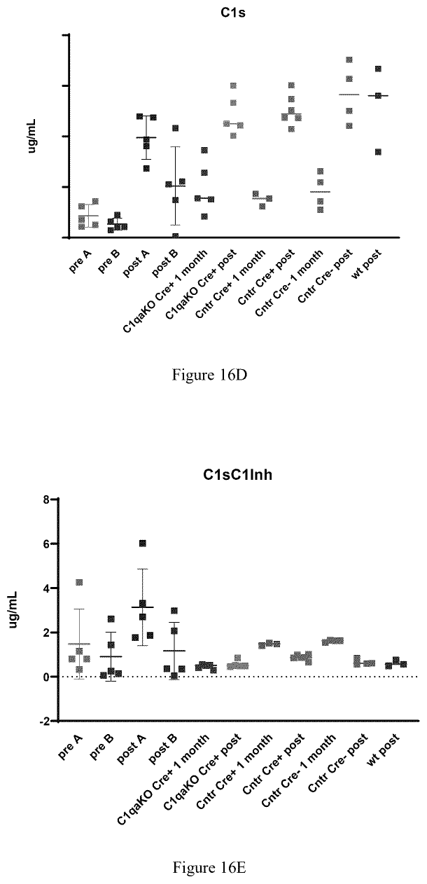

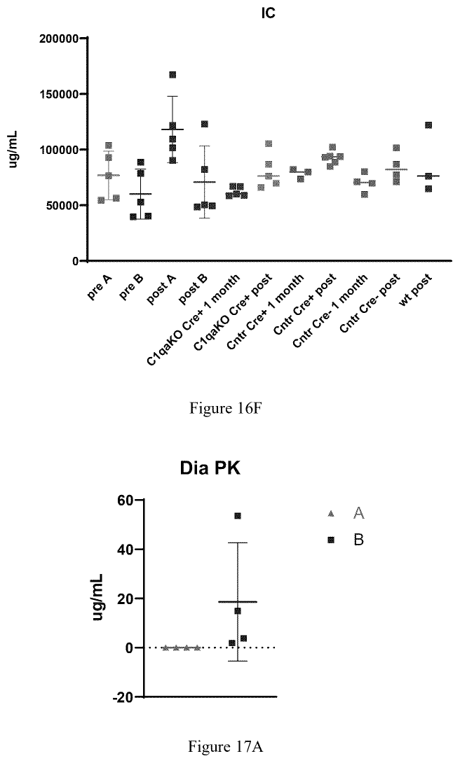

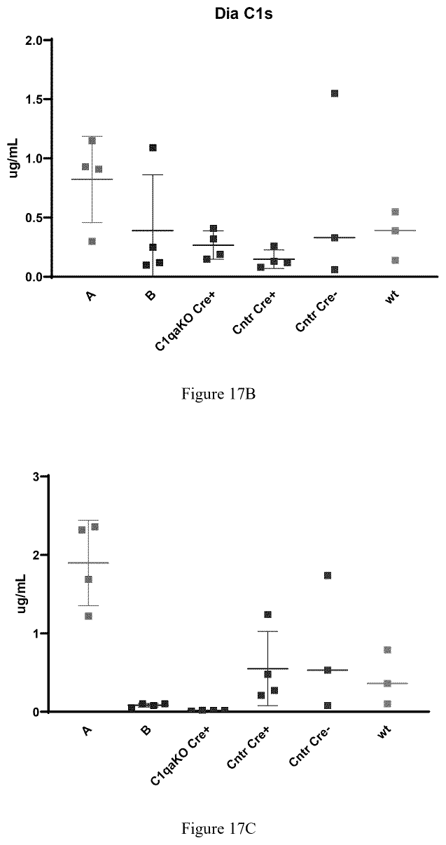

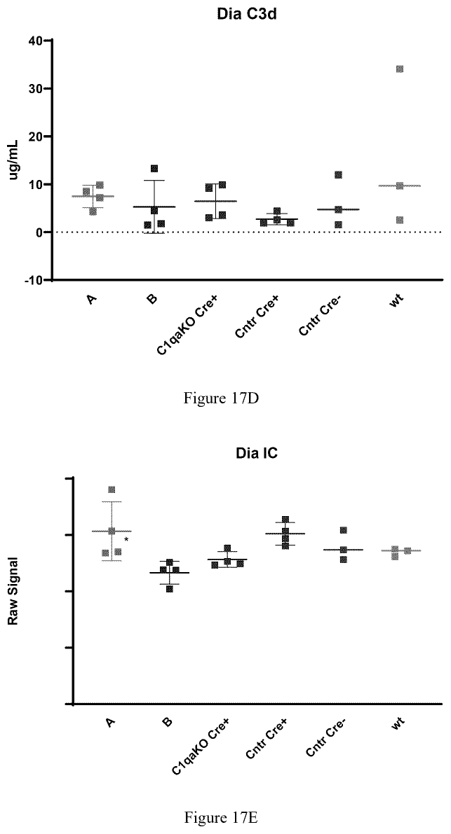

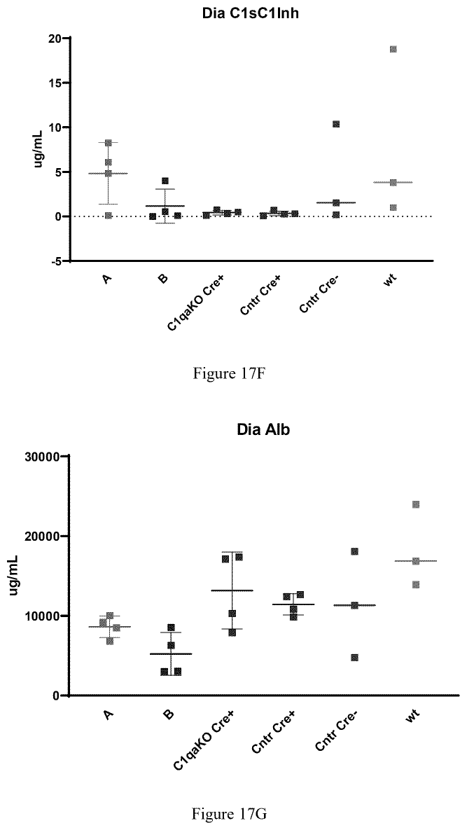

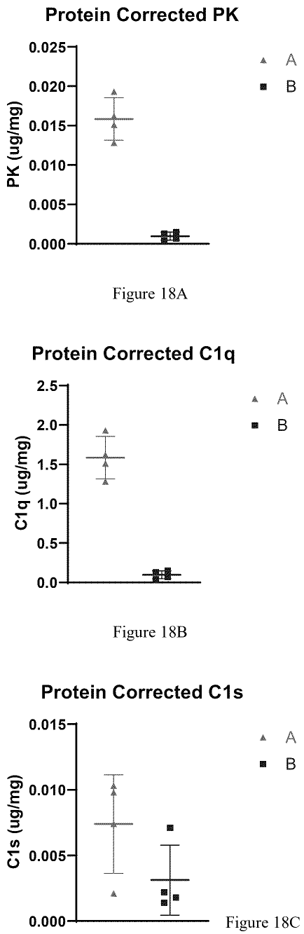

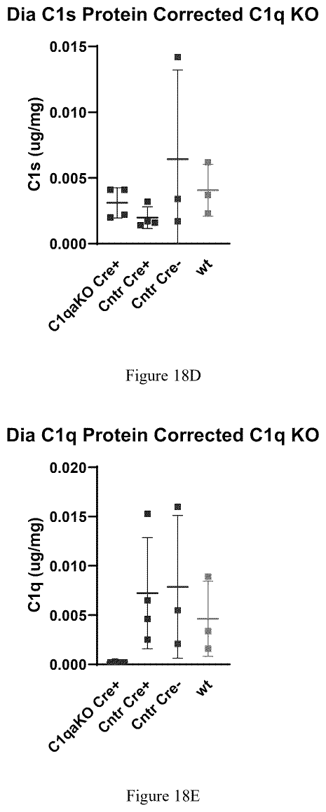

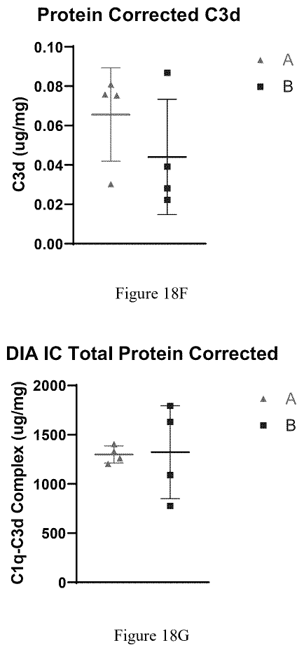

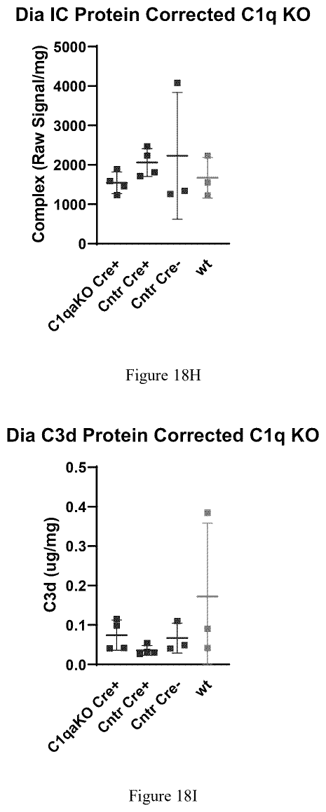

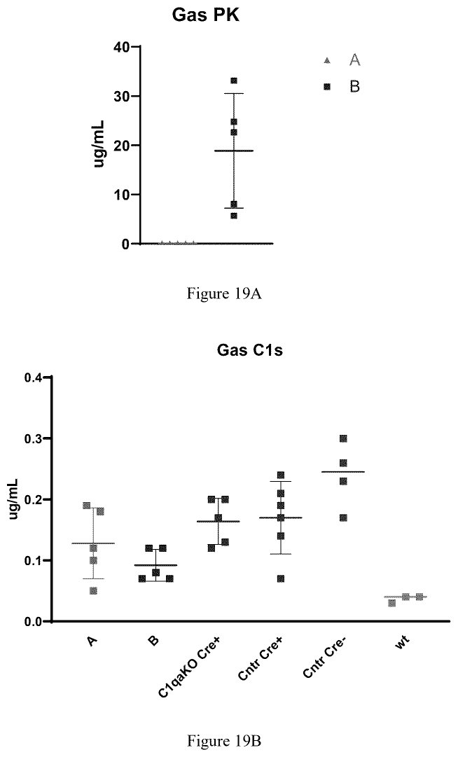

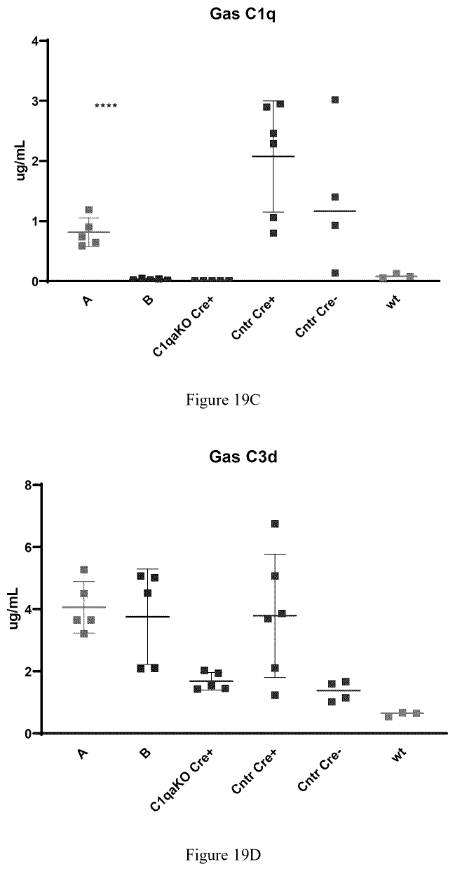

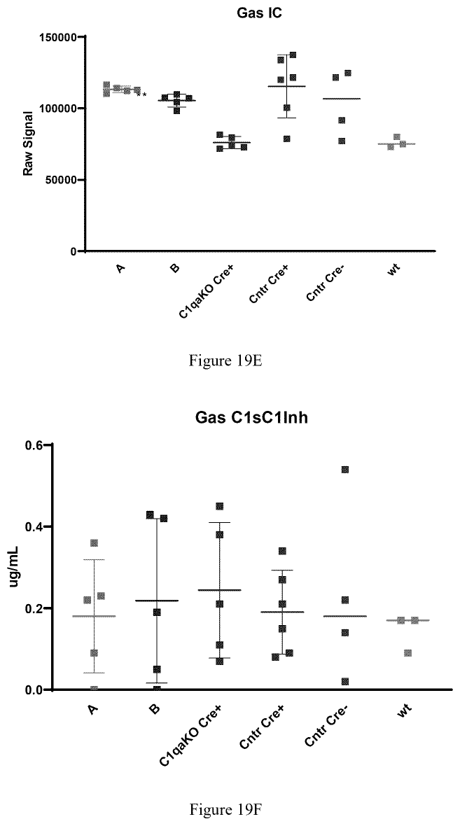

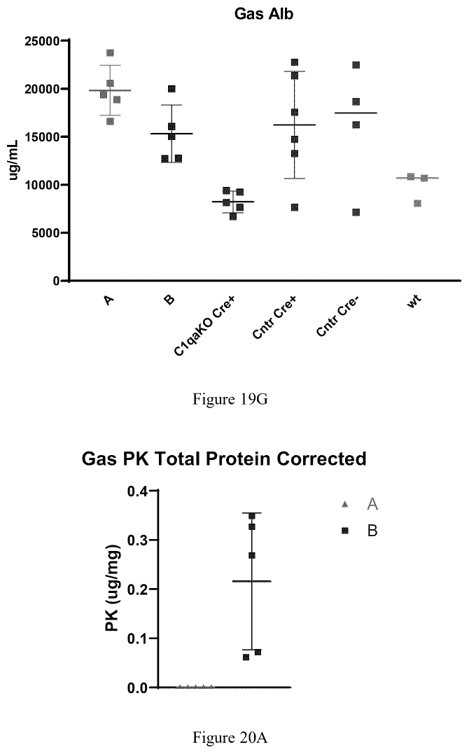

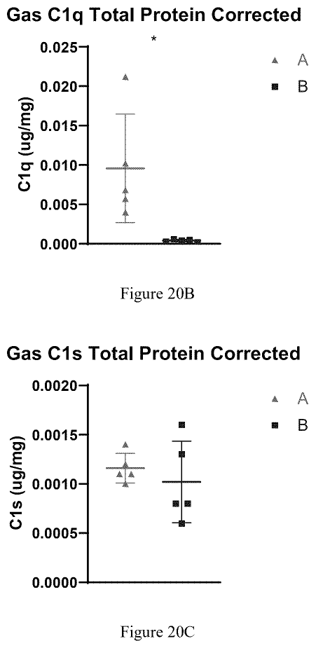

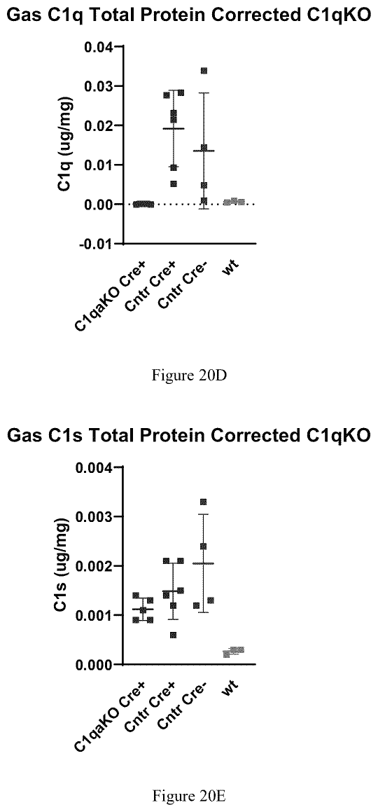

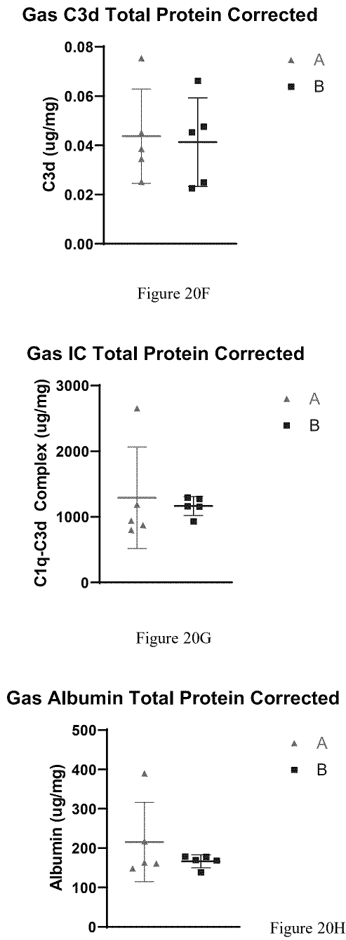

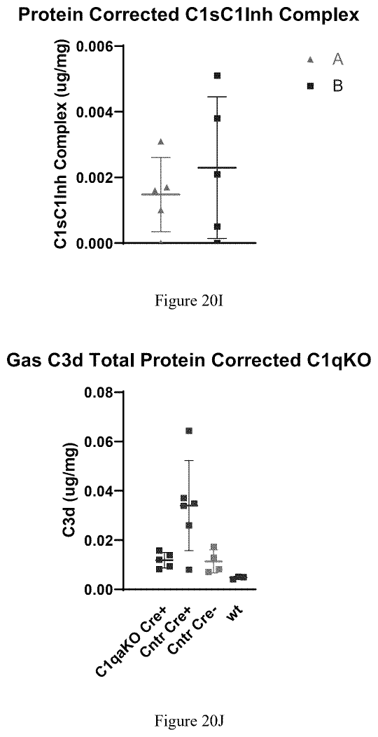

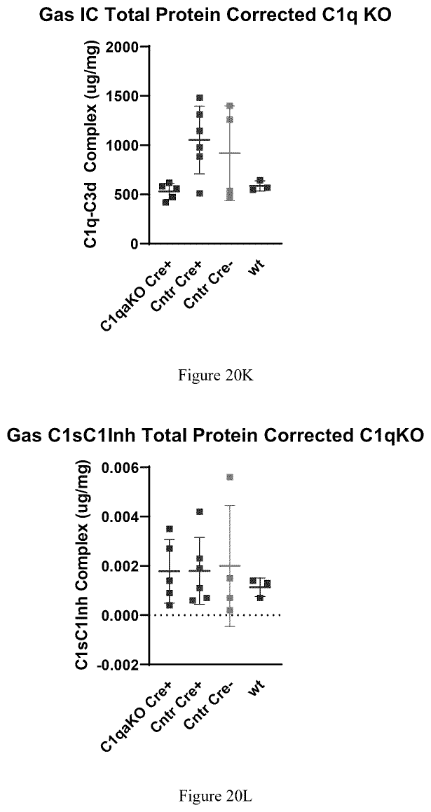

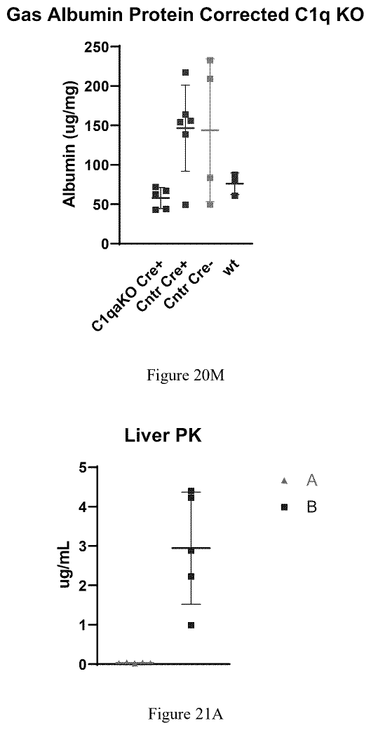

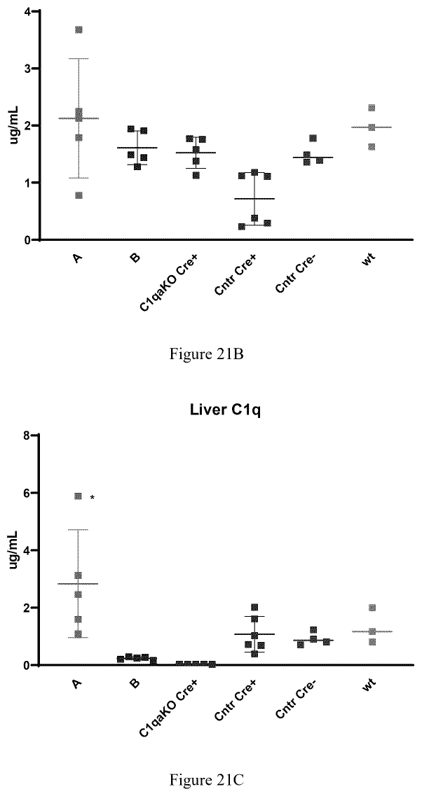

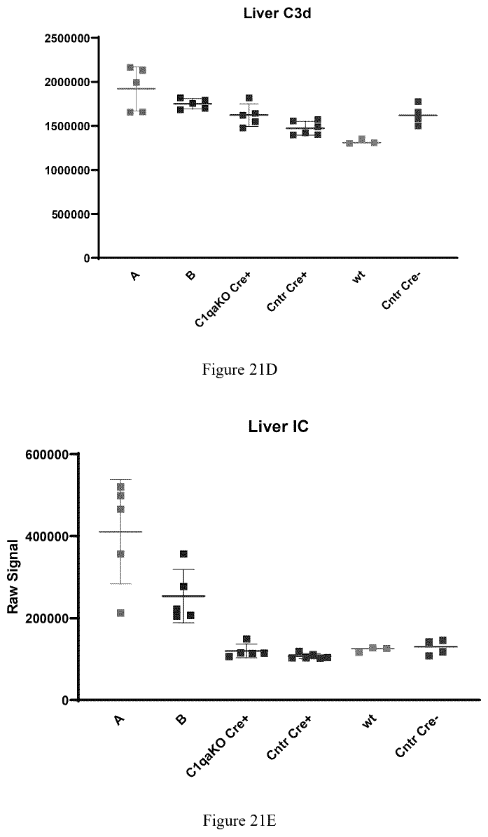

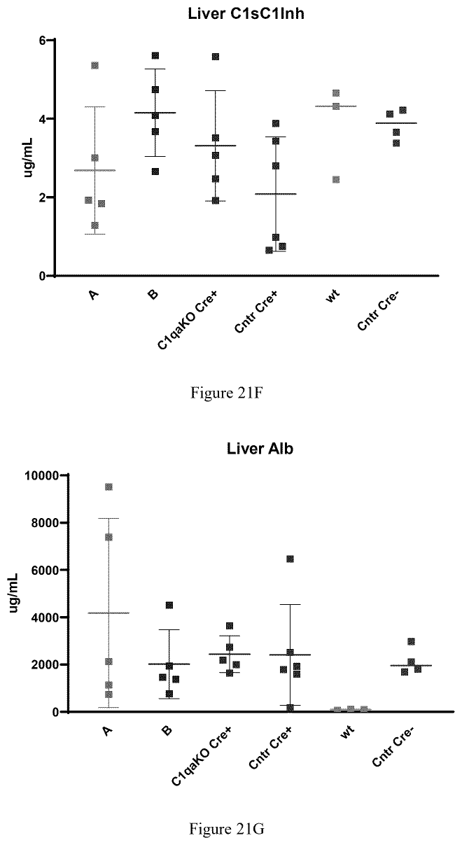

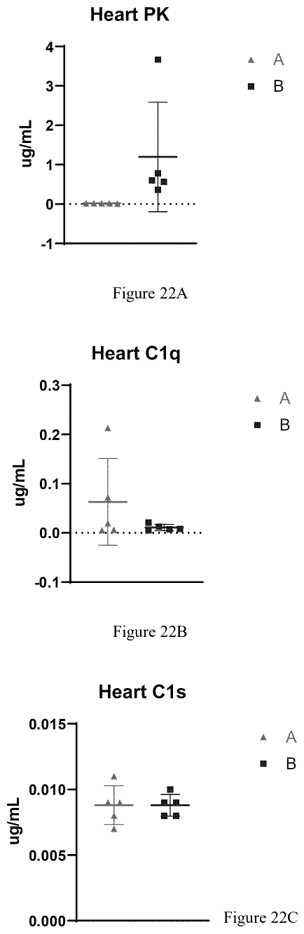

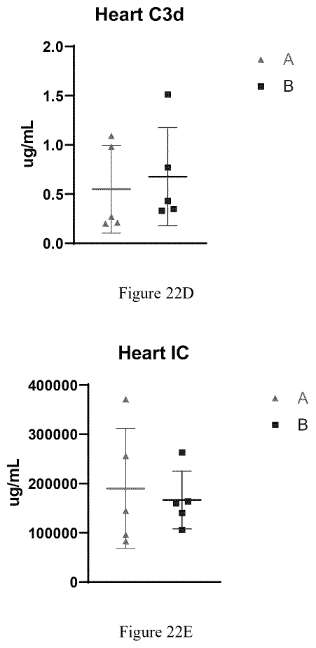

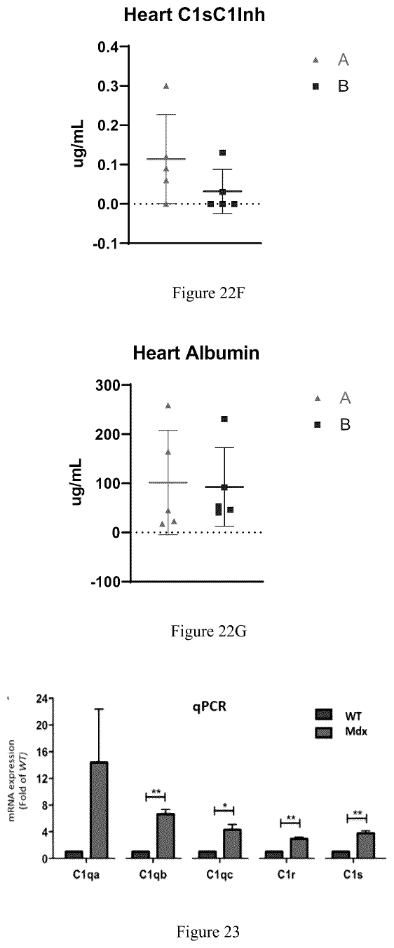

FIGS. 16-22: With respect to the anti-C1q blocking antibody treatment, the drug levels found in all samples collected from the mice treated with the anti-C1q blocking antibody (i.e., plasma, diaphragm, gastrocnemius, liver and heart) were increased compared to the mice treated with the control antibody. Within the analyzed samples, the gastrocnemius was the muscle with the highest drug's amount, while the heart was the muscle with the lowest drug's amount. The levels of C1q protein were reduced after the treatment with anti-C1q blocking antibody in all the analyzed samples. In particular, C1q depletion was strongly effective in the diaphragm, in the gastrocnemius and in the liver, in which C1q levels in the samples collected from mice treated with the anti-C1q blocking antibody were similar to the levels detected in the samples collected from the C1q genetically ablated mice (i.e., C1q^{KO};mdx^{4Cv}).

Overall, no differences were observed in the levels of other tested proteins (i.e., C3d, C1s) and proteins complexes (i.e., IC, C1sC1inh) in samples collected from the dystrophic mice treated with anti-C1q blocking antibody compared to the controls and in the C1q^{KO};mdx^{4Cv} samples compared to the controls.

### Example 9: Evaluation of Complement Levels in the Skeletal Muscles of Wild Type and Dystrophic

The levels of C1 complex subunits: C1qa, C1qb, C1qc, C1r, and C1s were evaluated by measuring the mRNA expression of ˜1 year old wild type and mdx^{4Cv} hindlimb muscles. C1qa and C1qb expression was measured in macrophages isolated from ˜1 year old wild type and mdx^{Cv} hindlimb muscles.

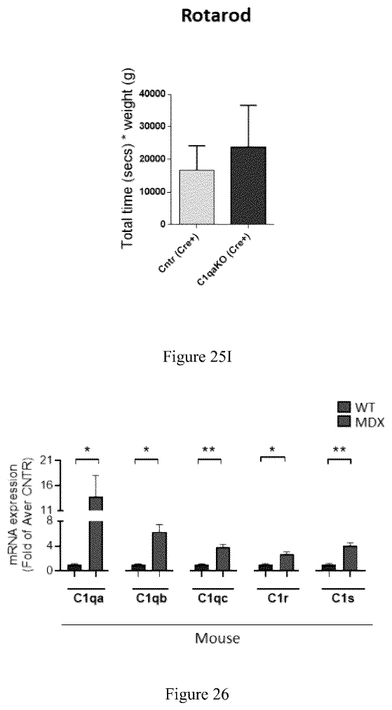

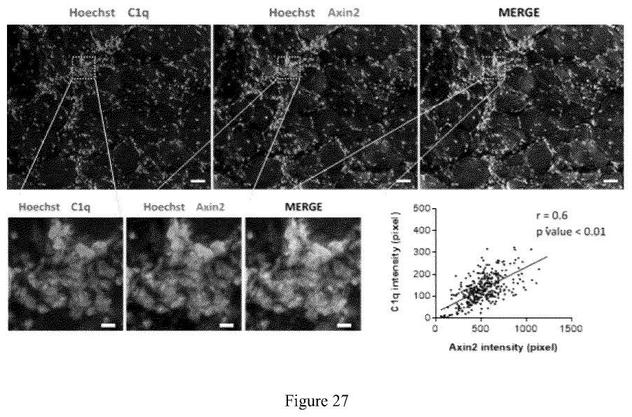

FIGS. 23 and 26: C1 complex components' expression is enhanced in dystrophic muscles. mRNA expression of C1 complex subunits C1qa, C1qb, C1qc, C1r, C1s was measured in the hindlimb muscles of ˜1 year old wild type (WT) and mdx^{4Cv} (Mdx) mice.

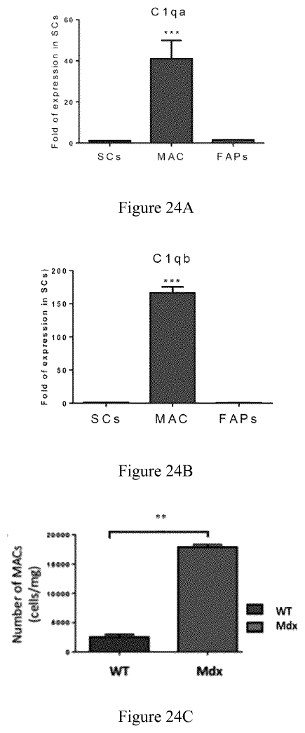

FIGS. 24A-24C: C1q subunits are expressed by infiltrating macrophages in the skeletal muscle of dystrophic mice. FIGS. 24A-24B show qPCR analysis of C1qa (A) and C1qb (B) expression in satellite cells (SC), Macrophages (MAC) and Fibro/Adipogenic Progenitors (FAPs) isolated from hindlimb muscles of ˜1 year old wild type (WT) and mdxCv (Mdx). FIG. 24C shows number of macrophages per mg of tissue in hindlimb muscles of ˜1 year old wild type (WT) and mdx^{4Cv} (Mdx) mice.

### Example 10: Behavioral Test in C1qa^{KO}. mdx^{4Cv} Mice and in Dystrophic Mice Treated with Anti-C1q Blocking Antibody

Behavioral tests were performed on C1qa^{KO};mdx^{4Cvl mice at ˜}1 month, ˜2 months and ˜3 months of age in order to evaluate the maximal hanging time before exhaustion (through the four limb hanging wire test), the total distance travelled, the mean speed, the percentage of mobile time (through the open field test), the two limbs grip strength, and the speed of the rotating rod.

The same behavioral test were performed on the Pax7^{CreER};R26R^{YFP};mdx^{4Cv} mice treated with anti-C1q blocking antibody or with the control antibody before and after the treatment.

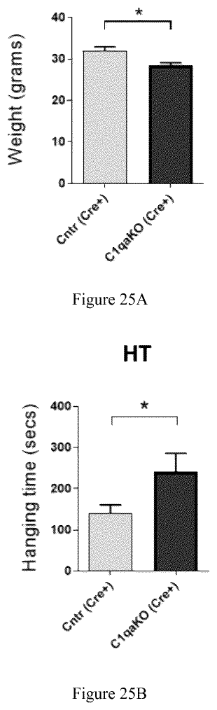

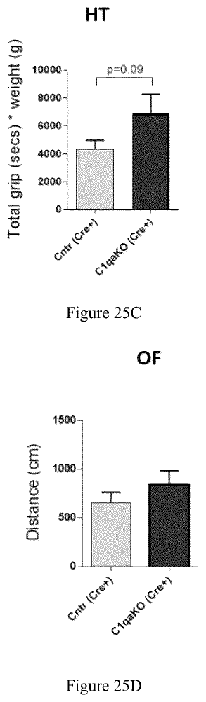

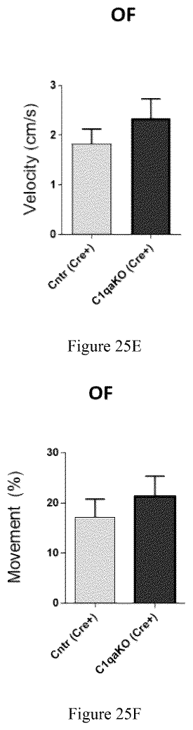

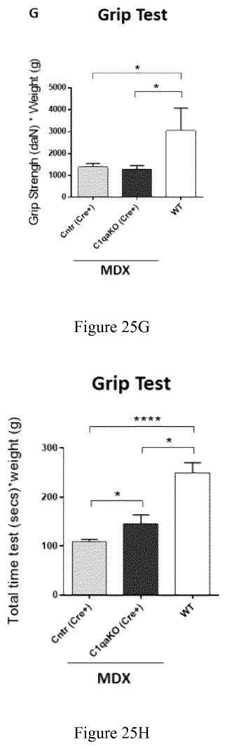

FIGS. 25A-25I show behavioral test on ˜1 year-old C1qa^{KO};mdx^{4Cv} mice and controls. FIG. 25A shows mice weight (grams) of ˜1 year old Lyz^{Cre+/-}C1qa^{FL/FL};mdx^{4Cv}(C1qaKO) and Lyz^{Cre+/-}C1qa^{WT/WT};mdx^{4Cv} (CNTR). FIGS. 25B, 25C show hanging test (HT) performed on mice as in (FIG. 25A). The total hanging time (FIG. 25B) and the total hanging time normalized for the mice weight (FIG. 25C) were evaluated. FIGS. 25D-25F show open field (OF) test performed on mice as in (FIG. 25A). The total distance (cm) (FIG. 25D), the mean speed (cm/sec) (FIG. 25E) and the percentage of time mobile (FIG. 25F) were evaluated. FIG. 25G, 25H show the two limbs grip test performed on mice as in (FIG. 25A). The maximal strength normalized for the mice weight (FIG. 25G) and the total grip time normalized for the mice weight (FIG. 25H) were evaluated. FIG. 25I shows the rotarod test performed on mice as in (FIG. 25A). The total walking time normalized for the mice weight was evaluated. N=5 (C1qaKO), N=7 (CNTR), N=2 (WT). Data are expressed as mean with SEM. Two-tailed unpaired t-test was applied. p >0.05: ns; p≤0.05: *; p≤0.01: **; p≤0.001: *** and p≤0.0001: ****.

### INCORPORATION BY REFERENCE

Each of the patents, published patent applications, and non-patent references cited herein are hereby incorporated by reference in their entirety.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of preventing, reducing risk of developing, slowing or blocking progression of, or treating Duchenne muscular dystrophy, Becker muscular dystrophy, a Limb-Girdle Muscular Dystrophy (LGMD), a Collagen Type VI-Related Disorder, a Congenital Muscular Dystrophy (CMD) or Congenital Myopathy, or a Distal Muscular Dystrophy/Myopathy, comprising administering to a subject an inhibitor of the classical complement pathway.

2-4. (canceled)

5. The method of claim 1, wherein the inhibitor of the classical complement pathway is a C1 complex inhibitor.

6-10. (canceled)

11. The method of claim 1, wherein the inhibitor of the classical complement pathway is a C1q inhibitor.

12. The method of claim 11, wherein the C1q inhibitor is an antibody, a peptide, a protein, a nucleic acid, a small molecule, a gene editing agent, a base editing agent, or an epigenetic editing agent.

13. (canceled)

14. The method of claim 12, wherein the antibody is an anti-C1q antibody.

15-20. (canceled)

21. The method of claim 1, wherein the antibody specifically binds to and neutralizes a biological activity of C1q.

22. The method of claim 21, wherein the biological activity is (1) C1q binding to an autoantibody, (2) C1q binding to C1r, (3) C1q binding to C1s, (4) C1q binding to IgM, (5) C1q binding to phosphatidylserine, (6) C1q binding to pentraxin-3, (7) C1q binding to C-reactive protein (CRP), (8) C1q binding to globular C1q receptor (gC1qR), (9) C1q binding to complement receptor 1 (CR1), (10) C1q binding to beta-amyloid, (11) C1q binding to calreticulin, (12) C1q binding to apoptotic cells, or (13) C1q binding to B cells.

23. The antibody of claim 21, wherein the biological activity is (1) activation of the classical complement activation pathway, (2) activation of antibody and complement dependent cytotoxicity, (3) CH50 hemolysis, (4) synapse loss, (5) B-cell antibody production, (6) dendritic cell maturation, (7) T-cell proliferation, (8) cytokine production (9) microglia activation, (10) immune complex formation, (11) phagocytosis of synapses or nerve endings, (12) activation of complement receptor 3 (CR3/C3) expressing cells or (13) neuroinflammation.

24-26. (canceled)

27. The method of claim 14, wherein the antibody is a monoclonal antibody, a polyclonal antibody, a recombinant antibody, a humanized antibody, a human antibody, a chimeric antibody, a monovalent antibody, a multispecific antibody, an antibody fragment, or antibody derivative thereof.

28. The method of claim 27, wherein the antibody is an antibody fragment and the antibody fragment is a Fab fragment, a Fab′ fragment, a F(ab′)2 fragment, a Fv fragment, a diabody, or a single chain antibody molecule.

29. The method of claim 14, wherein the antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7.

30. The method of claim 14, wherein the antibody comprises a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11.

31. The method of claim 14, wherein the antibody comprises a light chain variable domain comprising an amino acid sequence with at least about 95% homology to the amino acid sequence selected from SEQ ID NO: 4 and 35-38 and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7.

32. The method of claim 31, wherein the light chain variable domain comprising an amino acid sequence selected from SEQ ID NO: 4 and 35-38.

33. The method of claim 14, wherein the antibody comprises a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11.

34. The method of claim 33, wherein the heavy chain variable domain comprising an amino acid sequence selected from SEQ ID NO: 8 and 31-34.

35. The method of claim 14, wherein the antibody is an antibody fragment comprising a heavy chain Fab fragment of SEQ ID NO: 39 and a light chain Fab fragment of SEQ ID NO: 40.

36. The method of claim 1, wherein the inhibitor of the classical complement pathway is a C1r inhibitor.

37-45. (canceled)

46. The method of claim 1, wherein the inhibitor of the classical complement pathway is a C1s inhibitor.

47-55. (canceled)

56. A method of preventing, reducing risk of developing, slowing or blocking progression of, or treating Duchenne muscular dystrophy, comprising administering to a subject an inhibitor of the classical complement pathway.

57. A method of preventing, reducing risk of developing, slowing or blocking progression of, or treating Duchenne muscular dystrophy, Becker Muscular Dystrophy, a Limb-Girdle Muscular Dystrophy (LGMD), a Collagen Type VI-Related Disorder, a Congenital Muscular Dystrophy (CMD) or Congenital Myopathy, or a Distal Muscular Dystrophy/Myopathy, comprising administering to a subject a C1q inhibitor antibody, wherein the antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7, and a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11.
